(19) United States
(12) Patent Application Publication
(10) Pub. No.: US 2009/0012546 A1
(43) Pub. Date: Jan. 8, 2009

(54) DEVICES FOR TREATING GASTROESOPHAGEAL REFLUX DISEASE AND HIATAL HERNIA, AND METHODS OF TREATING GASTROESOPHAGEAL REFLUX DISEASE AND HIATAL HERNIA USING SAME

(75) Inventors: Colleen Stack N'diaye, Hillsborough, NC (US); Jeffrey A. Smith, Petaluma, CA (US); Geoffrey A. Orth, Sebastopol, CA (US); Michael S. Williams, Santa Rosa, CA (US); William L. Athas, Chapel Hill, NC (US); Thomas B. Miller, Perkiomenville, PA (US)

Correspondence Address: MYERS BIGEL SIBLEY & SAJOVEC, PO BOX 37428, RALEIGH, NC, 27627, US

(73) Assignee: SyneCor, LLC

(21) Appl. No.: 12/144,990

(22) Filed: Jun. 24, 2008

Related U.S. Application Data
(60) Provisional application No. 60/958,303, filed on Jul. 3, 2007.

(51) Int. Cl. A61B 17/122

(52) U.S. Cl. 606/157

(57) ABSTRACT

A device for treating GERD includes a pair of sections. Each section includes a proximal end portion with an arcuate interior opening, a distal end portion with an arcuate interior opening, an exterior plate portion, and a tapered body portion extending from the proximal end portion to the distal end portion. The pair of sections surround the esophagus at the gastro-esophageal junction thereof and to be secured together to form a tubular housing with a proximal orifice, a distal orifice smaller than the proximal orifice, and a plate adjacent the distal orifice. A portion of the housing adjacent the distal orifice is configured to elastically deform to constrict the esophagus and serve the function of the LES when a force is applied to the plate by the fundus. The proximal end portion of each mating section includes a flange portion configured to be attached to a body cavity wall.

### RELATED APPLICATION

This application claims the benefit of and priority to U.S. Provisional Patent Application No. 60/958,303, filed Jul. 3, 2007, the disclosure of which is incorporated herein by reference as if set forth in its entirety.

### FIELD OF THE INVENTION

The present invention relates generally to the treatment of gastroesophageal reflux disease and hiatal hernia, more specifically, to implantable devices for treating gastroesophageal reflux disease and hiatal hernia.

### BACKGROUND OF THE INVENTION

Gastroesophageal reflux disease (GERD) is caused by excessive reflux of gastric contents into the esophagus. GERD affects approximately 20% of Americans, with half of these suffering daily symptoms. Reflux esophagitis, injury to the esophageal mucosa, occurs in approximately 50% of patients with GERD; more serious complications occur in a minority.

The typical symptom of GERD is heartburn, particularly after meals and upon reclining. Symptom severity does not correlate with degree of tissue damage; patients with only mild symptoms can develop severe esophagitis. Other “typical” though less common symptoms include regurgitation (reflux of gastric contents into back of mouth) or dysphagia. Atypical manifestations include asthma, chronic cough, chronic laryngitis, sore throat, and noncardiac chest pain. GERD appears to be a causative, or at least an exacerbating, factor in up to 50% of such patients. Atypical manifestations continue to be underdiagnosed, although such presentations are being recognized with increasing frequency.

The gastroesophageal junction provides a natural barrier against excess reflux of gastric contents. A competent lower esophageal sphincter (LES) depends on four factors: the intrinsic LES pressure, the intra-abdominal location of the sphincter, extrinsic compression of the sphincter by the crural diaphragm, and an acute angle of His (between the distal esophagus and the cardiac stomach). Hiatal hernia displaces the LES above the diaphragm, thus decreasing its competency and contributing to reflux. Hiatal hernia also delays clearance of gastric content, promoting more severe esophagitis particularly Barrett's esophagus. Refluxed gastric content is cleared by esophageal peristalsis, neutralization by swallowed saliva (pH >6), and gravity. Factors that contribute to GERD pathogenesis include: incompetent LES, hiatal hernia, impaired esophageal peristalsis, delayed gastric emptying, gastric acid hypersecretion, and bile reflux.

Complications of GERD include Barrett's esophagus and peptic stricture, each of which affects approximately 10% of people with chronic GERD. In Barrett's esophagus, the normal epithelium of the esophagus (squamous) is replaced with metaplastic epithelium characteristic of the gastric or intestinal lining (columnar, with goblet cells). Columnar metaplasia may be of three types: gastric cardiac, gastric fundic, and intestinal. Intestinal metaplasia is associated with an increased risk of neoplasia; most cases of esophageal adenocarcinoma occur in the setting of Barrett's esophagus. Peptic stricture causes progressive dysphagia of solid food over months to years; 90% of patients can be successfully treated with esophageal dilatation with weighted bougies, graduated catheterizations, or balloon expansion.

Medical therapies for GERD include H₂-receptor antagonists, proton pump inhibitors, and prokinetic agents. Surgical treatment is typically recommended for patients who either require continuous or increasing dosages of medications and are good surgical patients, or in whom continuous PPI therapy is not desirable. Failure of medical therapy can also be an indication for surgery, although care must be exercised to rule out other causes.

A common surgical procedure for treating GERD is fundoplication, wherein the fundus (upper part) of the stomach is wrapped, or plicated, around the inferior part of the esophagus, thereby preventing the reflux of gastric acid. In a Nissen fundoplication, also called complete fundoplication, the fundus is wrapped all the way around the esophagus. Fundoplication is often done laparoscopically. When used as a method to alleviate gastroesophageal reflux symptoms in patients with delayed gastric emptying, this procedure is frequently done in conjunction with modification of the pylorus via pyloromyotomy or pyloroplasty. Fundoplication prevents or reduces reflux by three mechanisms: increasing LES pressure, accentuating angle of His, and increasing the length of the intra-abdominal esophagus.

Various surgical procedures for treating GERD are performed endoscopically. In one procedure, a series of stitches are placed in the lower esophagus to create a pleat in the sphincter. This pleat alters the gate or valve to reduce the backflow of acid from the stomach up through the esophagus. In another procedure, a full-thickness plication is created at the gastroesophageal junction under direct endoscopic visualization; enhancing the competency of the gastric cardia and restoring the normal anti-reflux barrier. Another endoscopic procedure uses electrodes to create tiny burns on the LES. When the burns heal, the scar tissue helps toughen the muscle, thereby improving the barrier function of the LES and reducing the frequency and severity of reflux.

Unfortunately, known complications are present with each of these procedures. Moreover, many patients find it necessary to continue drug treatment after each of these surgical procedures. As such, more successful options for treating GERD are desired.

### SUMMARY

In view of the above discussion, a device for treating GERD and methods of implanting same are provided. These devices are configured to prevent the reflux of gastric contents into the esophagus that otherwise might occur because of an incompetent LES.

According to some embodiments of the present invention, a device for treating GERD is configured to be positioned around the esophagus of a patient adjacent to the fundus, and includes a pair of mating sections. Each mating section includes a proximal end portion with an arcuate interior opening, a distal end portion with an arcuate interior opening, an exterior plate portion, and a tapered body portion extending from the proximal end portion to the distal end portion. The pair of mating sections are configured to surround the esophagus at the gastro-esophageal junction thereof and to be secured together in abutting relationship to form a tubular housing with a proximal orifice, a distal orifice smaller than the proximal orifice, and a plate adjacent the distal orifice. A portion of the housing adjacent the distal orifice is configured to elastically deform to constrict the esophagus and serve the function of the LES when a force is applied to the plate by the fundus (i.e., when the stomach fills with food). The proximal end portion of each mating section includes a flange portion configured to be attached to a body cavity wall, such as the diaphragm.

The mating sections may be secured together in various ways. In some embodiments, the mating sections are secured together via a bio-compatible adhesive. In other embodiments, the mating sections are secured together via one or more snap connectors. In other embodiments, the mating sections are secured together via one or more fasteners (e.g., screws, bolts, rivets, clips, t-bar connectors, sutures, etc.). In some embodiments, the mating sections are hingedly connected to facilitate implantation within a patient and to eliminate implantation of separate components.

A method of treating GERD, according to some embodiments of the present invention, includes securing a pair of mating sections together around a portion of an esophagus of a patient at the gastro-esophageal junction thereof and adjacent the fundus, and attaching a proximal end portion thereof to a body cavity wall via, for example, surgical clips, sutures, staples, and/or adhesive. Each mating section includes a proximal end portion with an arcuate interior opening, a distal end portion with an arcuate interior opening and an exterior plate portion, and a body portion extending from the proximal end portion to the distal end portion. The pair of mating sections are configured to surround the esophagus at the gastro-esophageal junction thereof and to be secured together in abutting relationship to form a tubular housing with a proximal orifice, a distal orifice that may be smaller than the proximal orifice, and a plate adjacent the distal orifice. The plate may rest on the fundus, may be spaced apart slightly from the fundus, or may be secured to the fundus.

A portion of the housing adjacent the distal orifice is configured to elastically deform to constrict the esophagus and serve the function of the LES when a force is applied to the plate by the fundus. The proximal end portion of each mating section includes a flange portion configured to be attached to a body cavity wall, such as the diaphragm.

According to other embodiments of the present invention, a device for treating GERD is configured to be positioned around the esophagus of a patient adjacent to the fundus. The device includes a tubular housing with opposite proximal and distal orifices, and a plate adjacent the distal orifice that extends outwardly from the housing. A portion of the housing adjacent the distal orifice is configured to elastically deform to constrict the esophagus when a force is applied to the plate by the fundus. The device includes a slit in the housing that allows the device to be wrapped around the esophagus. Once secured around the esophagus, the housing is secured together, for example, via fasteners.

According to other embodiments of the present invention, a device for treating GERD is configured to be positioned around the esophagus of a patient adjacent to the fundus. The device includes a tubular housing with opposite proximal and distal orifices and an aperture in a side thereof. A plate is pivotally engaged with the housing adjacent the distal orifice, and is pivotable between a first position and second position. A flap is attached to the plate and is configured to extend within the housing aperture to constrict the esophagus when the plate is moved to the second position by the fundus. In some embodiments, the flap includes a raised portion or rib that contacts the esophagus when the plate is moved to the second position. In some embodiments, the rib may be an interchangeable component that can be inserted and removed from the flap. Moreover, the interchangeable rib may be adjustable to allow a surgeon to adjust the amount of closure on the esophagus.

GERD treatment devices according to embodiments of the present invention may be implanted around an esophagus via surgery, via a laparoscopy tool, via an endoscope, and via a NOTES (Natural Orifice Translumenal Endoscopic Surgery) device inserted down the esophagus of a patient.

Embodiments of the present invention described herein are designed to prevent reflux by the same three mechanisms as fundoplication; increasing LES pressure, accentuating the angle of His, and increasing the length of the intra-abdominal esophagus. Implantable devices, according to embodiments of the present invention, can remain stable, and because they are attached to the diaphragm instead of to the stomach, they should not erode through the gastrointestinal (GI) tissue. Moreover, the implantation procedure can be performed laparoscopically in a simpler procedure than the Nissen fundoplication.

Embodiments of the present invention are particularly effective in the treatment of hiatal hernia. Each of the various GERD treatment devices described herein act as a lock to prevent hiatal hernia. Moreover, the flange at the proximal end of the various GERD treatment devices described herein, and which are configured to be attached to the diaphragm of a patient, help prevent and/or repair the occurrence of hiatal hernia.

### BRIEF DESCRIPTION OF THE DRAWINGS

The accompanying drawings, which form a part of the specification, illustrate embodiments of the present invention. The drawings and description together serve to fully explain the invention.

FIG. 1 is a schematic illustration of a human stomach and a portion of the small intestine.

FIG. 2 illustrates the gastro-esophageal junction region of a human stomach.

FIG. 3 illustrates a device for treating GERD, according to embodiments of the present invention, implanted around a portion of an esophagus and adjacent the fundus.

FIG. 4 is an enlarged, perspective view of the GERD treatment device of FIG. 3.

FIG. 5 is a cross-sectional view of the GERD treatment device of FIG. 3 taken along lines 3-3.

FIG. 6 is an exploded, perspective view of the GERD treatment device of FIG. 3, illustrating the mating sections.

FIG. 7 illustrates the elastic deformation of the tubular housing of the GERD treatment device of FIG. 3 adjacent the distal orifice when a force is applied to the plate by the fundus.

FIG. 8 is a perspective view of a device for treating GERD, according to other embodiments of the present invention.

FIG. 9 is a perspective view of a device for treating GERD, according to other embodiments of the present invention.

FIG. 10 is a perspective view of a device for treating GERD, according to other embodiments of the present invention.

FIG. 11 is a perspective view of a GERD treatment device, according to other embodiments of the present invention.

FIG. 12 is a cross-sectional view of the GERD treatment device of FIG. 11 taken along lines 11-11.

FIG. 13 illustrates the GERD treatment device of FIG. 11 implanted around a portion of an esophagus and adjacent the fundus.

FIG. 14A is a perspective view of a device for treating GERD, according to other embodiments of the present invention.

FIG. 14B is a cross-sectional view of the device of FIG. 14A taken along lines 14B-14B.

FIG. 15A is a perspective view of a device for treating GERD, according to other embodiments of the present invention.

FIG. 15B is a cross-sectional view of the device of FIG. 15A taken along lines 15B-15B.

FIG. 16A illustrates a device for treating GERD, according to other embodiments of the present invention.

FIG. 16B is a cross-sectional view of the device of FIG. 16A taken along lines 16B-16B.

FIG. 17 is a perspective view of a device for treating GERD, according to other embodiments of the present invention.

FIG. 18A is a perspective view of a device for treating GERD, according to other embodiments of the present invention.

FIG. 18B is a cross-sectional view of the device of FIG. 18A taken along lines 18B-18B.

FIG. 18C is a top perspective view of the device of FIG. 18A.

FIG. 19 is a perspective view of a device for treating GERD, according to other embodiments of the present invention.

FIG. 20A is a cross-sectional view of a device for treating GERD, according to other embodiments of the present invention.

FIG. 20B is a cross-sectional view of the device of FIG. 20A taken along lines 20B-20B.

FIG. 21 is a cross-sectional view of a device for treating GERD, according to other embodiments of the present invention.

FIG. 22 is a top perspective view of a device for treating GERD, according to other embodiments of the present invention.

FIG. 23 is a perspective view of a device for treating GERD, according to other embodiments of the present invention.

FIG. 24 is a perspective view of a device for treating GERD, according to other embodiments of the present invention.

FIG. 25 is a bottom perspective view of a device for treating GERD, according to other embodiments of the present invention.

FIG. 26 is a top plan view of the device of FIG. 25.

FIGS. 27-32 are perspective views of a device for treating GERD, according to other embodiments of the present invention.

FIGS. 33-37 illustrate fasteners for securing a device for treating GERD around an esophagus, according to embodiments of the present invention.

FIGS. 38-39, 40A-40B, 41A-41B, 42A-42B, 43A-43B and 44A-44B illustrate tools for securing the fasteners of FIGS. 33-37, according to embodiments of the present invention.

FIG. 45 is a perspective view of a device for treating GERD, according to other embodiments of the present invention.

FIG. 46 is a cross-sectional view of the device of FIG. 45 taken along lines 46-46.

FIG. 47 illustrates the device of FIG. 46 with a raised portion on the flap thereof.

FIG. 48 illustrates the implantation of a GERD treatment device, according to an embodiment of present invention, around an esophagus via a NOTES device inserted down the esophagus of a patient.

### DETAILED DESCRIPTION

While the invention is susceptible to various modifications and alternative forms, specific embodiments thereof are shown by way of example in the drawings and will herein be described in detail. It should be understood, however, that there is no intent to limit the invention to the particular forms disclosed, but on the contrary, the invention is to cover all modifications, equivalents, and alternatives falling within the spirit and scope of the invention as defined by the claims. Like reference numbers signify like elements throughout the description of the figures.

As used herein, the singular forms “a,” “an,” and “the” are intended to include the plural forms as well, unless expressly stated otherwise. It should be further understood that the terms “comprises” and/or “comprising” when used in this specification are taken to specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In the drawings, the thickness of lines, layers and regions may be exaggerated for clarity. It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of a device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of “over” and “under”. A device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

It will be understood that, although the terms “first”, “second”, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a “first” element, component, region, layer or section discussed below could also be termed a “second” element, component, region, layer or section without departing from the teachings of the present invention.

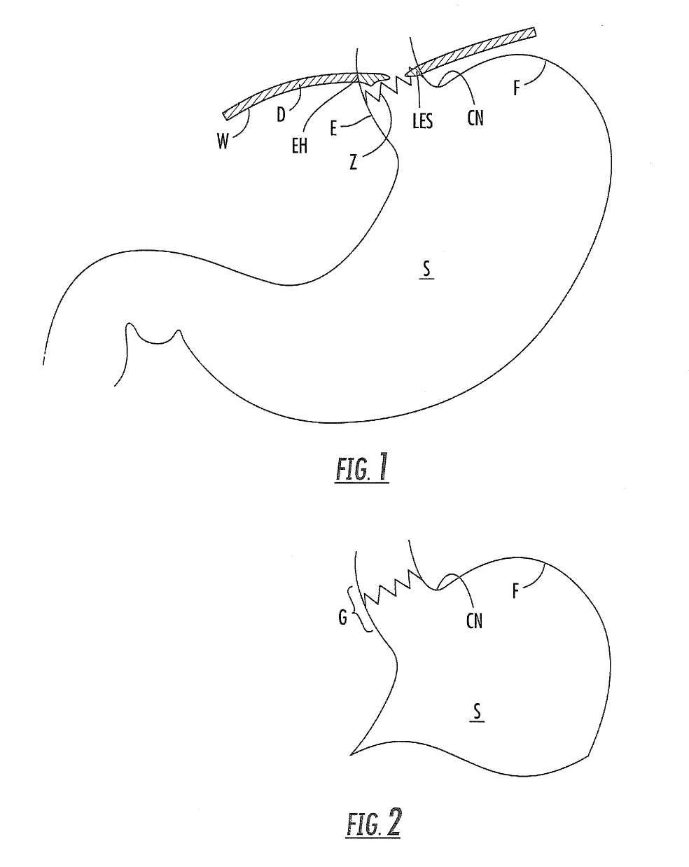

An anatomical view of a human stomach S and associated features is shown in FIGS. 1-2. The esophagus E delivers food from the mouth to the stomach S. The z-line or gastro-esophageal junction Z is the irregularly-shaped border between the thin tissue of the esophagus and the thicker tissue of the stomach wall. The gastro-esophageal junction region is the region encompassing the distal portion of the esophagus E, the z-line Z, and the proximal portion of the stomach S. The fundus F is an upper portion of the stomach S which joins with the esophagus E at an acute angle commonly referred to as the cardiac notch CN. The angle between the distal esophagus and the cardiac notch is referred to as the angle of His.

A lower esophageal sphincter (LES) is shown positioned within the esophagus E in the region of the diaphragm D. In FIG. 1, the LES is shown exaggerated for the purpose of illustration. The esophagus E passes through an esophageal hiatus EH in the diaphragm D. The LES normally provides control of reflux of contents of the stomach S into the esophagus E. However, in patients with severe GERD, the LES does not function correctly (i.e., the LES is incompetent).

Embodiments of devices for treating GERD are described herein that include a structure secured around the esophagus at the gastro-esophageal junction region G (FIG. 2). These devices are configured to serve the function of the LES, preserve the Angle of His, repair hiatal hernia, and control reflux of contents of the stomach S into the esophagus E.

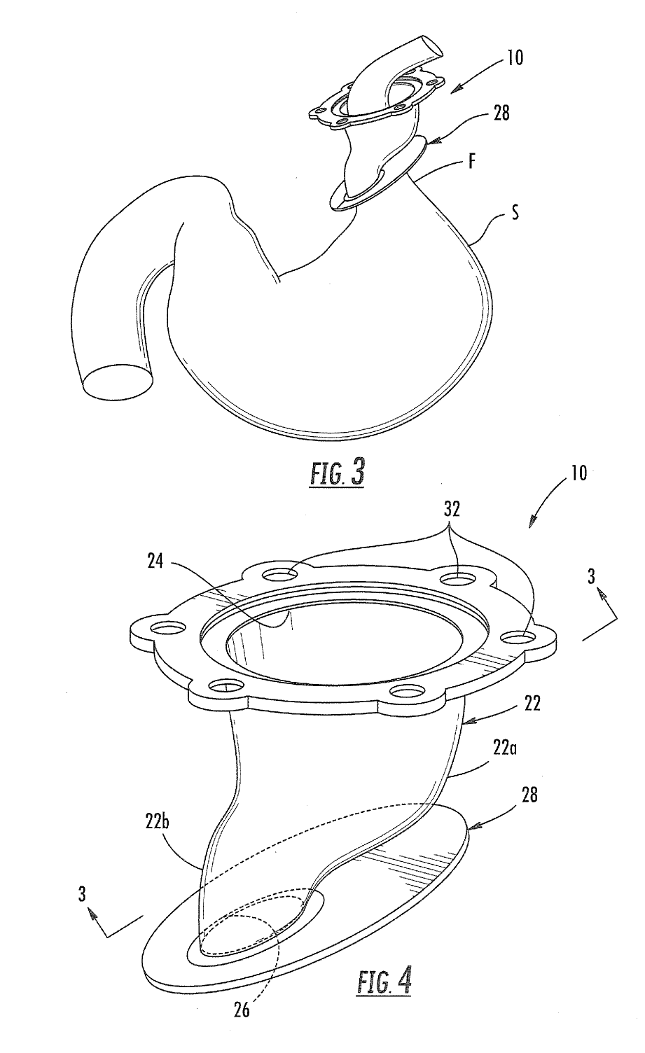

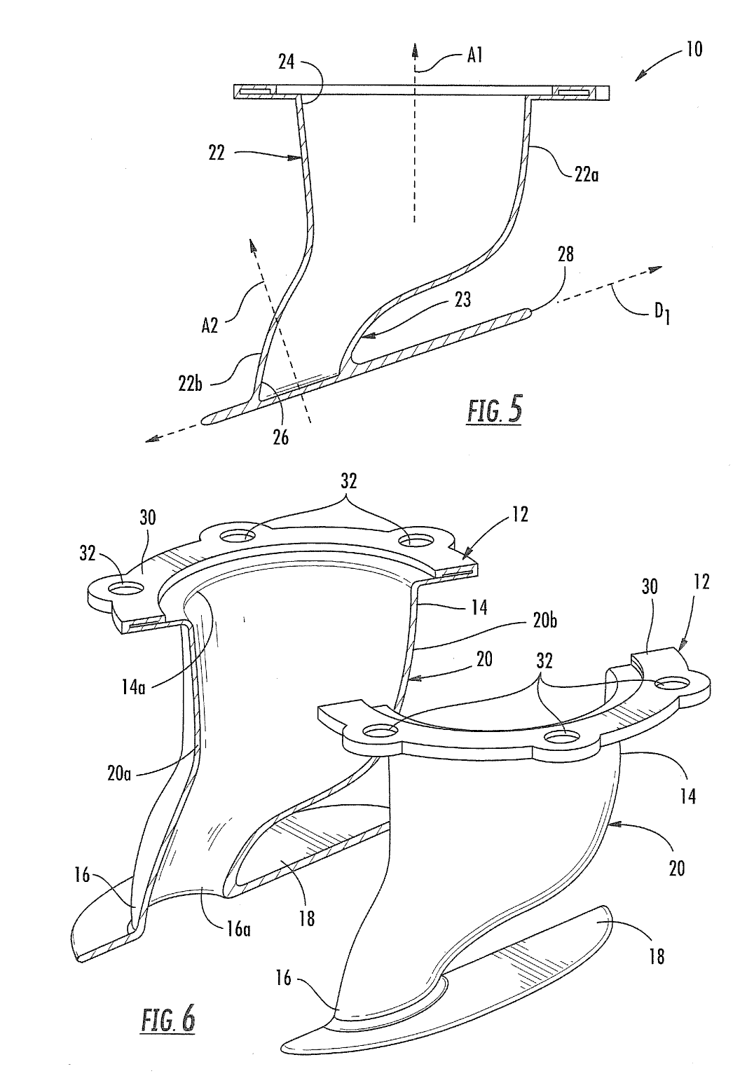

One embodiment of a device 10 for treating GERD, according to the present invention, is illustrated in FIGS. 3-7 and includes a pair of mating sections 12. Each mating section 12 includes a proximal end portion 14 with an arcuate interior opening 14a, a distal end portion 16 with an arcuate interior opening 16a, an exterior plate portion 18, and a tapered body portion 20 extending from the proximal end portion 14 to the distal end portion 16. The pair of mating sections 12 are configured to surround the esophagus at the gastro-esophageal junction thereof and to be secured together in abutting relationship. When secured together, the mating sections 12 form a tubular housing 22 with a proximal orifice 24, a distal orifice 26, and a plate 28 adjacent the distal orifice 26 (FIGS. 3, 4). A portion 23 of the housing 22 adjacent the distal orifice 26 is configured to elastically deform to constrict the esophagus when a force is applied to the flange 28 by the fundus F (FIG. 7).

The illustrated tubular housing 22 has an upper portion 22a and a lower portion 22b. The upper housing portion 22a has a larger diameter than that of the lower housing portion 22b. In addition, the centerline of the distal orifice 24 is offset from the centerline of the proximal orifice 26, as illustrated in FIG. 5. Axial directions A₁ and A₂ indicate the centerlines of the proximal and distal orifices 24, 26, respectively, and show the offset. This offset configuration, along with the reduced diameter of the distal (lower) portion 22b of the tubular housing 22 causes the tubular housing 22 to deform at a neck portion 23 thereof as illustrated in FIG. 7 when a force is applied to the plate 28.

FIG. 4 is a perspective view of the GERD treatment device 10 illustrating the pair of mating sections 12 secured together in abutting relationship. The illustrated GERD treatment device 10 has a proximal orifice 24 with a generally circular configuration and a distal orifice 26 with a generally oval configuration. However, the proximal and distal orifices 24, 26 may have various configurations and are not limited to generally circular and oval configurations, respectively. For example, the proximal orifice 24 may have an elongated, oval configuration, the distal orifice 26 may have a circular configuration, etc. Moreover, the proximal and distal orifices 24, 26 may have cross-sectional configurations selected for the particular esophagus of a patient. The proximal orifice 24 generally has a cross-sectional area of between about 0.05 square centimeters (0.05 cm²) and about 20 square centimeters (20 cm²), and the distal orifice 26 has a cross-sectional area of between about 0.05 square centimeters (0.05 cm²) and about 20 square centimeters (20 cm²). However, embodiments of the present invention are not limited to these cross-sectional area ranges for the proximal orifice 24 and distal orifice 26.

In some embodiments, the size of the proximal and/or distal orifices 24, 26 may be increased or decreased, for example by removing or installing spacers or inserts within the arcuate interior openings 14a, 16a of the mating sections 12, or by selecting mating sections 12 with different proximal and/or distal arcuate interior openings 14a, 16a and/or different proximal and/or distal end configurations. This enables a physician implanting the GERD treatment device 10 to set the proximal and/or distal orifices to a size appropriate for a patient. In some cases, it will also allow the physician to make adjustments to the proximal and/or distal orifices 24, 26 after the GERD treatment device 10 has been implanted. For example, the size of the proximal and/or distal orifices may be adjustable remotely (i.e., transabdominally) after implantation (e.g., mechanically, magnetically, pneumatically, hydraulically, and/or telemetrically via RF energy or ultrasound, etc.). Alternatively, GERD treatment devices, according to embodiments of the present invention, may be custom-made for a patient.

As illustrated in FIG. 5, the proximal orifice 24 defines a first axial direction A₁, and the distal orifice 26 defines a second axial direction A₂ that is different from the first axial direction. The plate 28 (which comprises joined plate portions 18) is elongated in a direction that is substantially transverse to the first and second axial directions A₁ and A₂. In the illustrated embodiment, the plate 28 is elongated in a direction D₁ that is substantially orthogonal to the second axial direction A₂.

The illustrated plate 28 has a generally elongated, oval configuration. However, plate 28 may have various shapes and configurations and is not limited to the illustrated configuration. Moreover, the plate 28 may extend in various directions relative to the first and second axial directions A₁ and A₂. When the GERD treatment device 10 is implanted within a patient, the plate 28 may rest on the fundus (F, FIG. 1) or may be spaced apart slightly from the fundus. Moreover, in some embodiments, the plate 28 may be attached to the fundus. The angle the plate 28 makes with the esophagus may be actively adjusted (e.g., via an inflatable member, balloon, etc.) to modify the Angle of HIS.

In the illustrated embodiment, the proximal end portion 14 of each mating section 12 includes a flange portion 30 that is configured to be attached to a body cavity wall W, for example the diaphragm (D, FIG. 1) of a patient. Flange portions 30 may have various configurations to facilitate attachment of the GERD treatment device 10 to a body cavity wall and need not have the illustrated configuration. The illustrated flange portions 30 include a plurality of spaced-apart apertures 32 which are provided to facilitate attachment of the GERD treatment device 10 to a body cavity wall W (e.g., the diaphragm D, FIG. 1) via fasteners inserted therethrough and through or in a body cavity wall W. In some embodiments, the flange portions 30 may be reinforced, for example via insert molding wherein a reinforcing element (e.g., a grommet, etc.) is positioned within the material of the flange portion 30.

However, the apertures 32 are not required. In some embodiments, the material of the flange portions 30 may be selected such that fasteners can be inserted therethrough without the need for apertures. Exemplary fasteners for attaching the GERD treatment device 10 to a body cavity wall include, but are not limited to, sutures, clips, t-bar connectors, etc. In addition, the GERD treatment device 10 may be attached to a body cavity wall via bio-compatible adhesives.

The flange apertures 32 may have various configurations and numbers without limitation. Although each flange portion 30 is illustrated as having three apertures 32, more apertures or fewer apertures may be utilized. Moreover, the flange portions 30 may be reinforced in the locations of the apertures 32. Each flange portion 30 and/or area surrounding an aperture 32 may be made of a suitably dense radio-opaque material, such as titanium, gold, or barium to add in visualization of the GERD treatment device 10 during or after the implantation within a patient. Each flange portion 30 and/or area surrounding an aperture 32 may also be marked using a different color to facilitate identification and orientation of fasteners.

In some embodiments of the present invention, the mating sections 12 may be secured together via bio-compatible adhesive. In other embodiments, the mating sections 12 may be secured together via one or more snap connectors. In other embodiments, the mating sections 12 may be secured together via one or more fasteners (e.g., screws, bolts, rivets, clips, t-bar connectors, sutures, etc.).

In some embodiments, the two mating sections 12 may be hingedly connected, rather than being separate components, so as to be movable relative to each other between open and joined configurations. For example, the two mating sections 12 may be hingedly connected at one or more locations along the edge portions 20a or 20b of the respective body portions 20. Such a configuration may facilitate implantation within a patient as a single component, rather than as individual components. Moreover, a hinge connection between the two mating sections 12 may facilitate accurate alignment of the two mating sections 12 when joined together around a portion of the esophagus of a patient.

The illustrated mating sections 12 are substantially identical (mirrored halves). However, embodiments of the present invention are not limited to identical mating sections. A mating section 12 can have a different shape, size, and/or configuration from another mating section(s) 12, according to other embodiments of the present invention.

In use, when the fundus F of a patient's stomach bulges upwardly as a result of the ingestion of food, the fundus F imparts a force on the plate 28, as illustrated in FIG. 7. This force causes the plate 28 to pivot upwardly, as indicated by arrow A₃, which causes a neck portion 23 of the tubular housing 22 adjacent the distal orifice 26 thereof to elastically deform. This elastic deformation constricts the esophagus and serves the function of the LES, thereby preventing or reducing reflux of gastric contents into the esophagus.

The illustrated mating sections 12 may be formed from various materials including, but not limited to polymeric materials, metals, fabric, mesh, and combinations thereof. Each mating section 12 may be entirely formed from the same material or different portions of each mating section 12 may be formed from different materials. In addition, one portion of each mating section 12 may be bioabsorbable and another portion of each mating section 12 may be permanent. Exemplary materials are described below.

According to some embodiments of the present invention, the illustrated GERD treatment device 10 may be configured to be removed from a patient. For example, the mating sections 12 may be joined together such that they can be separated from each other at a later point in time (i.e., the mating sections 12 are removably secured together). In other embodiments of the present invention, the illustrated GERD treatment device 10 may be designed to be permanently implanted within a patient.

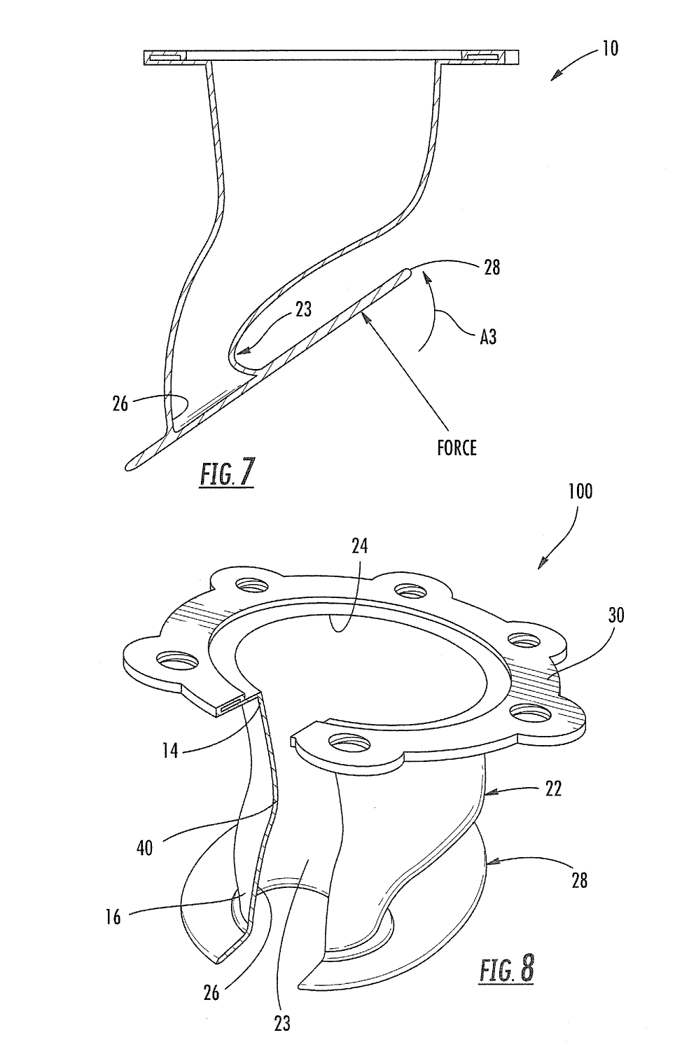

Referring to FIG. 8, a GERD treatment device 100, according to other embodiments of the present invention, is illustrated. The illustrated GERD treatment device 100 is a flexible, unitary device having a slit 40 in the housing 22 that allows the device 100 to be stretched open and wrapped around an esophagus. The illustrated GERD device 100, when wrapped around an esophagus forms a tubular housing 22 with a proximal orifice 24, a distal orifice 26, and a plate 28 adjacent the distal orifice 26. A portion 23 of the housing adjacent the distal orifice is configured to elastically deform to constrict the esophagus when a force is applied to the plate 28 by the fundus F, as described above. Once around an esophagus, the device 100 can be secured together via one or more fasteners (e.g., screws, bolts, rivets, clips, t-bar connectors, sutures, etc.) and/or via an adhesive as described above. The device 100 is anchored to the diaphragm via the flange 30, as described above.

FIGS. 9 and 10 illustrate various configurations for securing the device 100 of FIG. 8 together, according to some embodiments of the present invention, once wrapped around an esophagus. FIG. 9 illustrates a pair of fasteners 102 that are configured to secure the tubular housing 22 together around an esophagus. Each fastener 102 includes a pair of eyelets 104, one on each side of the slit 40 in the housing 22. A pin 106 with an enlarged head is inserted through the two eyelets 104 of each fastener 102 and latches the device 100. The device 100 can be unlatched and removed from an esophagus by removing the pins 106 from the respective eyelets 104 of each fastener 102. In some embodiments, the eyelets 104 may be reinforced, for example via insert molding wherein a reinforcing element (e.g., a grommet, etc.) is positioned within the material to form each eyelet 104.

FIG. 10 illustrates a plurality of pairs of eyelets 110 that are configured to secure the tubular housing 22 together around an esophagus. The eyelets in each pair are located on respective sides of the slit 40 and are aligned to receive a fastener (e.g., screws, bolts, rivets, clips, t-bar connectors, sutures, etc.) therethrough.

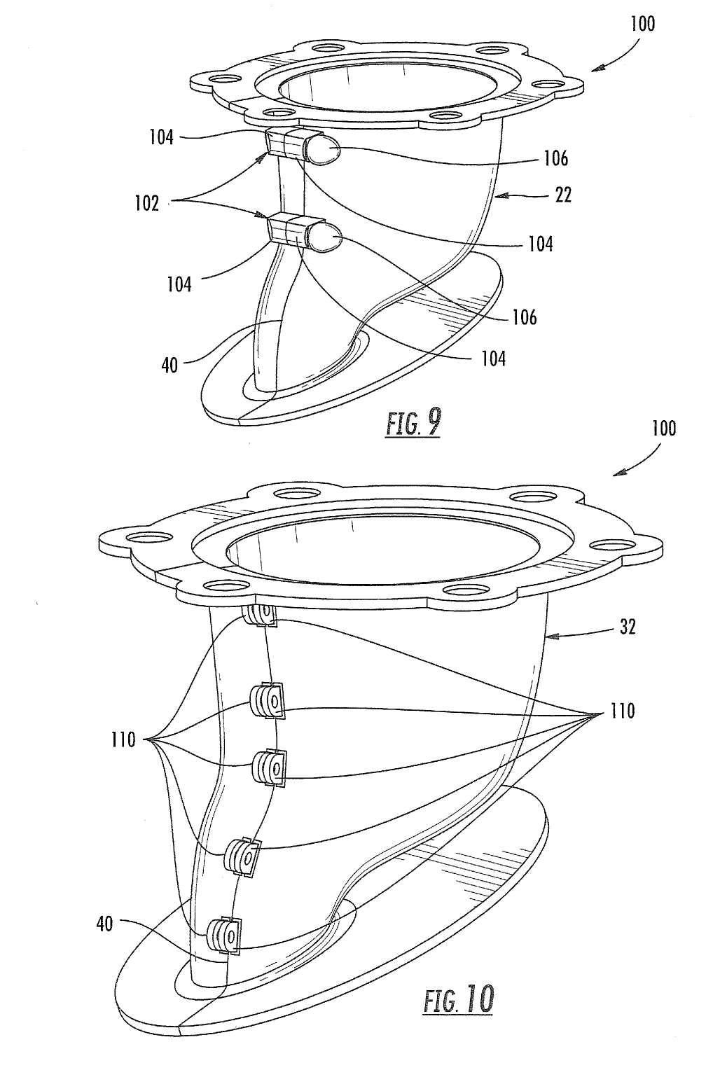

Another embodiment of a device for treating GERD, according to the present invention, is illustrated in FIGS. 11-13. The illustrated device 200 is similar in construction and function to the device 10 of FIGS. 3-7 with the exception of the configuration of the housing 222. The housing 222 of the illustrated device 200 has a tapered, straight configuration, rather than the tapered curved configuration of the device 10 of FIGS. 3-7. The device 200 may be a unitary device with a slit, similar to device 100 of FIG. 8. Alternatively, the device 200 may include a pair of mating sections, similar to device 10 of FIG. 3.

An exemplary mating section 212 is illustrated in FIG. 12 and includes a proximal end portion 214 with an arcuate interior opening 214a, a distal end portion 216 with an arcuate interior opening 216a, an exterior plate portion 218, and a tapered body portion 220 extending from the proximal end portion 214 to the distal end portion 216. A pair of the mating sections 212 are configured to surround the esophagus at the gastro-esophageal junction thereof and to be secured together in abutting relationship. When secured together, the mating sections 212 form a tubular housing 222 with a proximal orifice 224, a distal orifice 226, and a plate 228 adjacent the distal orifice 226. The illustrated tapered tubular housing 222 has an upper portion 222a and a lower portion 222b. The upper housing portion 222a has a larger diameter than that of the lower housing portion 222b. The device 200 is anchored to the diaphragm of a patient via the flange portion 230, as described above. A portion 223 of the housing 220 is configured to elastically deform to constrict the esophagus when a force is applied to the flange 228 by the fundus F, as described above.

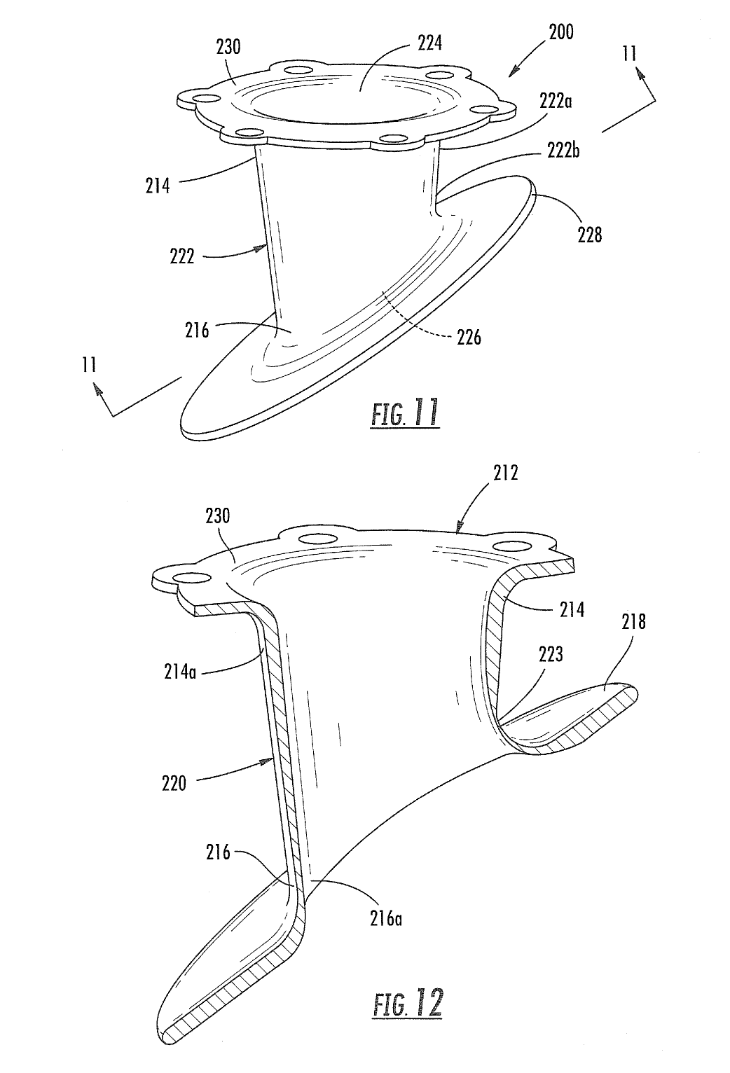

FIG. 13 illustrates the GERD treatment device of FIG. 11, implanted around a portion of an esophagus and adjacent the fundus F.

Another embodiment of a device for treating GERD, according to the present invention, is illustrated in FIGS. 14A-14B. The illustrated device 300 is similar in construction and function to the device 10 of FIGS. 3-7 with the exception that the device 300 is a flexible device having a longitudinally extending slit 40 in the housing 322 that allows the device 300 to be stretched open and wrapped around an esophagus. The illustrated GERD treatment device 300, when wrapped around an esophagus, forms a tubular housing 322 with a proximal orifice 324, a distal orifice 326, and a plate 328 adjacent the distal orifice 326. A portion 323 of the housing adjacent the distal orifice 326 is configured to elastically deform to constrict the esophagus when a force is applied to the plate 328 by the fundus F, as described above.

The illustrated GERD treatment device 300 includes an enlarged rib portion 340 at each slit edge. Each rib portion 340 includes a respective plurality of spaced-apart apertures 342. The spaced-apart apertures 342 are configured to align when the device 300 is wrapped around an esophagus such that fasteners (e.g., screws, bolts, rivets, clips, t-bar connectors, sutures, etc.) can be inserted therethrough to secure the rib portions 340 of device 300 together. The device 300 is anchored to the diaphragm via the flange portion 330, as described above.

In some embodiments, one or both of the rib portions 340 may be reinforced, for example via insert molding wherein a reinforcing element (e.g., a strip of strong material with apertures formed therein, plurality of grommets, etc.) is positioned within the material of the device to form the rib portions 340.

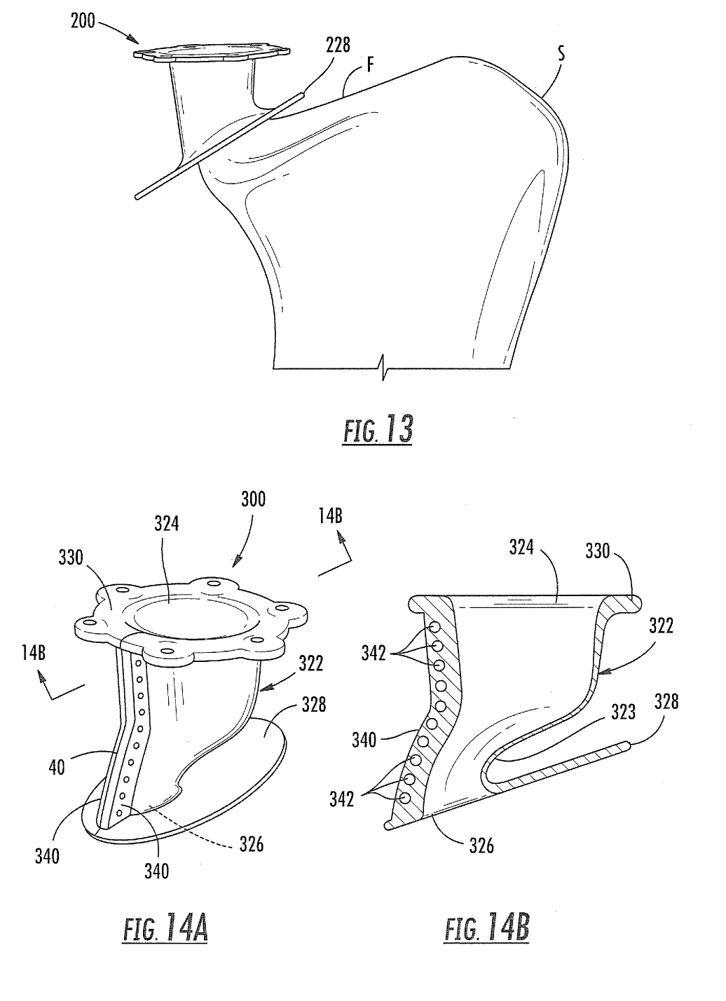

Another embodiment of a device for treating GERD, according to the present invention, is illustrated in FIGS. 15A-15B. The illustrated device 400 is similar in construction and function to the device 300 of FIGS. 14A-14B with the exception of the configuration of the housing 422. The housing 422 of the illustrated device 400 has a tapered, straight configuration, rather than the tapered curved configuration of the device 300 of FIGS. 14A-14B. The illustrated device 400 has a longitudinally extending slit 40 in the housing 422 that allows the device 400 to be stretched open and wrapped around an esophagus. The illustrated GERD-treatment device 400, when wrapped around an esophagus, forms a tubular housing 422 with a proximal orifice 424, a distal orifice 426, and a plate 428 adjacent the distal orifice 426. A portion 423 of the housing adjacent the distal orifice 426 is configured to elastically deform to constrict the esophagus when a force is applied to the plate 428 by the fundus F, as described above.

The illustrated GERD treatment device 400 includes an enlarged rib portion 440 at each slit edge. Each rib portion 440 includes a respective plurality of spaced-apart apertures 442. The spaced-apart apertures 442 are configured to align when the device 400 is wrapped around an esophagus such that fasteners (e.g., screws, bolts, rivets, clips, t-bar connectors, sutures, etc.) can be inserted therethrough to secure the rib portions 440 of the device 400 together. The device 400 is anchored to the diaphragm of a patient via the flange portion 430, as described above. Flange portion 430 includes a plurality of spaced-apart apertures 432 which are provided to facilitate attachment of the device 400 to a body cavity wall. In some embodiments, the periphery of the flange portion 430 may be reinforced, for example via insert molding wherein a reinforcing element (e.g., a grommet, etc.) is positioned within the material of the flange portion 430 to form each attachment aperture 432 in the flange portion 430.

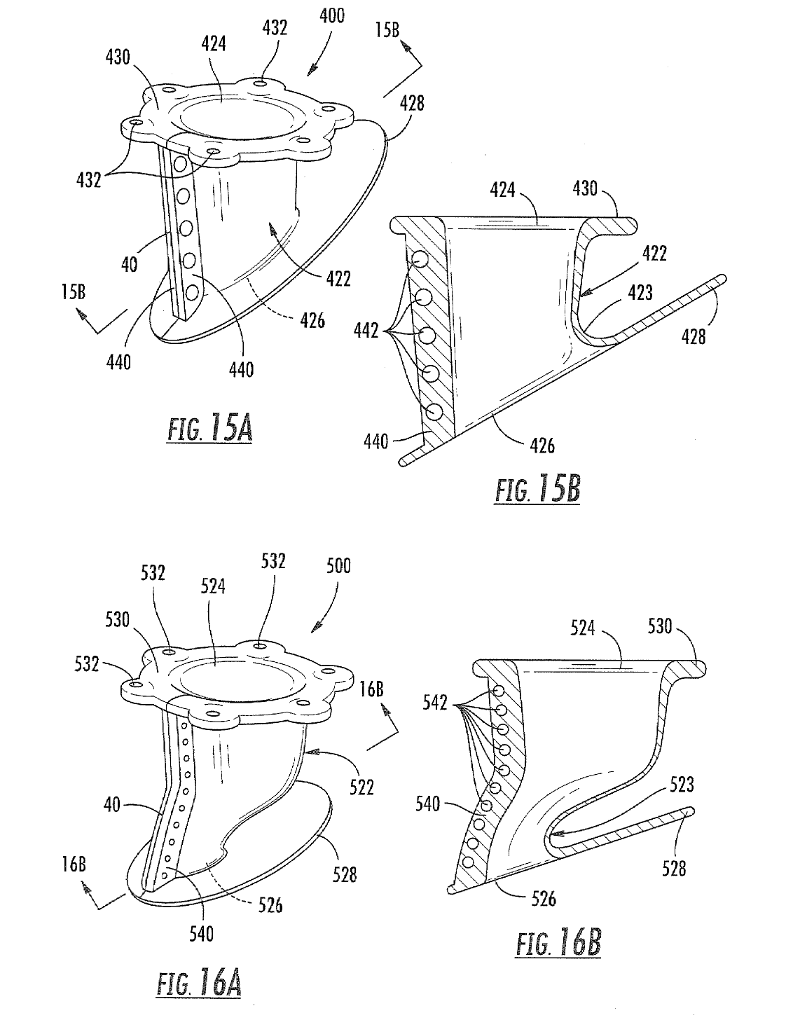

Another embodiment of a device for treating GERD, according to the present invention, is illustrated in FIGS. 16A-16B. The illustrated device 500 is similar in construction and function to the device 300 of FIGS. 14A-14B with the exception of the configuration of the housing 522. The illustrated device 500 has a longitudinally extending slit 40 in the housing 522 that allows the device 500 to be stretched open and wrapped around an esophagus. The illustrated GERD treatment device 500, when wrapped around an esophagus, forms a tubular housing 522 with a proximal orifice 524, a distal orifice 526, and a plate 528 adjacent the distal orifice 526. A portion 523 of the housing adjacent the distal orifice 526 is configured to elastically deform to constrict the esophagus when a force is applied to the plate 528 by the fundus F, as described above. The distal orifice 526 is larger than the distal orifice 326 of the device 300 of FIGS. 14A-14B.

The illustrated GERD treatment device 500 includes an enlarged rib portion 540 on each side of the slit 40. Each rib portion 540 includes a respective plurality of spaced-apart apertures 542. The spaced-apart apertures 542 are configured to align when the device 500 is wrapped around an esophagus such that fasteners (e.g., screws, bolts, rivets, clips, t-bar connectors, sutures, etc.) can be inserted therethrough to secure the rib portions 540 of the device 500 together. The device 500 is anchored to the diaphragm via the flange portion 530, as described above. In some embodiments, the periphery of the flange portion 530 may be reinforced, for example via insert molding wherein a reinforcing element (e.g., a grommet, etc.) is positioned within the material of the flange portion 530 to form each attachment aperture 532 in the flange portion 530.

Another embodiment of a device for treating GERD, according to the present invention, is illustrated in FIG. 17. The illustrated device 600 is similar in construction and function to the device 400 of FIGS. 15A-15B with the exception of the configuration of plate 628. The illustrated plate 628 includes a pair of slots 629 formed therein. These slots 629 can facilitate attachment of the plate 628 to the stomach via sutures and/or other types of fasteners. Sutures and other types of fasteners are easily inserted through the slots 629 and into the fundus F. In some embodiments, the peripheral region of the slots may be reinforced, for example via insert molding wherein a reinforcing element is positioned within the material of the plate 628 adjacent each slot 629. When the device 600 is secured to the diaphragm D of a patient via flange portion 630, attachment of plate 628 to the stomach can prevent the plate 628 and the device 600 from unwanted movement.

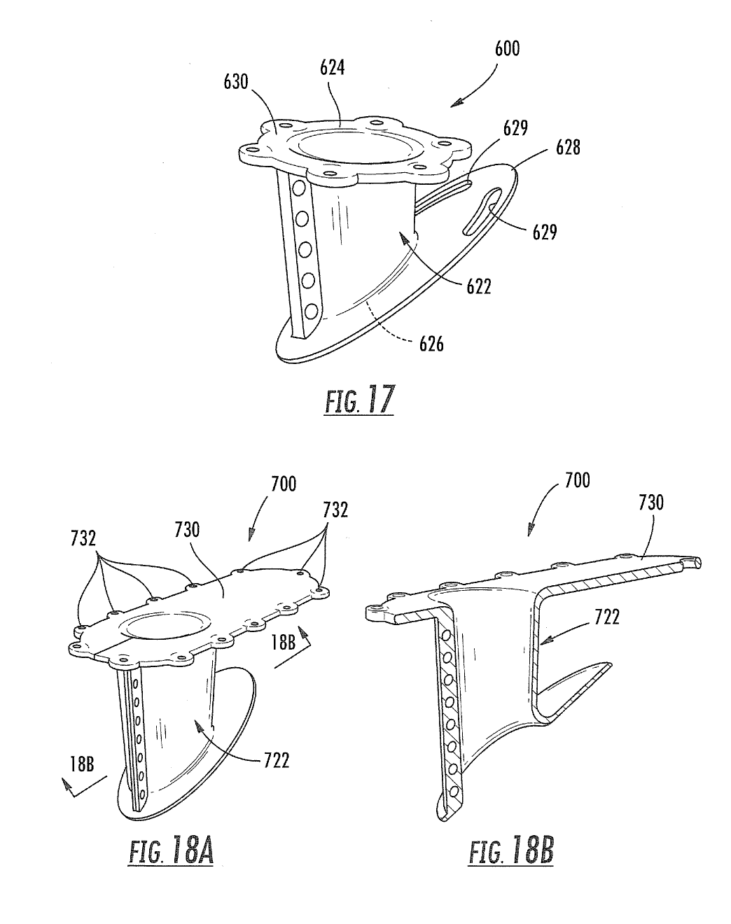

Another embodiment of a device for treating GERD, according to the present invention, is illustrated in FIGS. 18A-18C. The illustrated device 700 is similar in construction and function to the device 400 of FIGS. 15A-15B with the exception of the configuration of flange portion 730 and housing 722. The housing 722 of the illustrated device 700 has a tapered, tubular configuration, but is slightly longer than the housing 422 of device 400. In addition, flange portion 730 has an elongated configuration for attachment to a body cavity wall W, for example the diaphragm (D, FIG. 1) of a patient. Flange portion 730 includes a plurality of spaced-apart apertures 732 formed along the periphery thereof that facilitate attachment to the diaphragm D. In some embodiments, the periphery of the flange portion 730 may be reinforced, for example via insert molding wherein a reinforcing element (e.g., a grommet, etc.) is positioned within the material of the flange portion 730 to form each attachment aperture 732 in the flange portion 730.

FIG. 18B is a cross-sectional view of the device 700 taken along lines 18B-18B and illustrates the tapered configuration of the housing 722. FIG. 18C is another perspective view of the device 700 that illustrates the slot 40 that extends along one side of the device 700 and that allows the device 700 to be stretched open and wrapped around an esophagus.

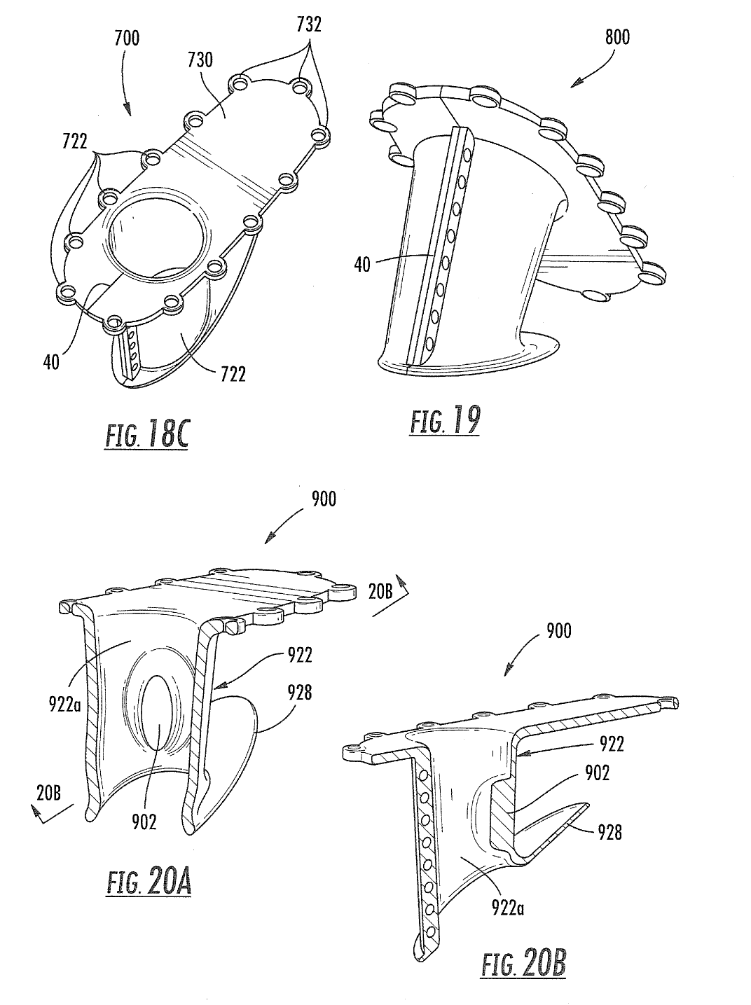

Another embodiment of a device for treating GERD, according to the present invention, is illustrated in FIG. 19. The illustrated device 800 is similar in construction and function to the device 700 of FIGS. 18A-18C with the exception of the location of the slit 40. The slit 40 is offset relative to a side of the housing 722, as illustrated.

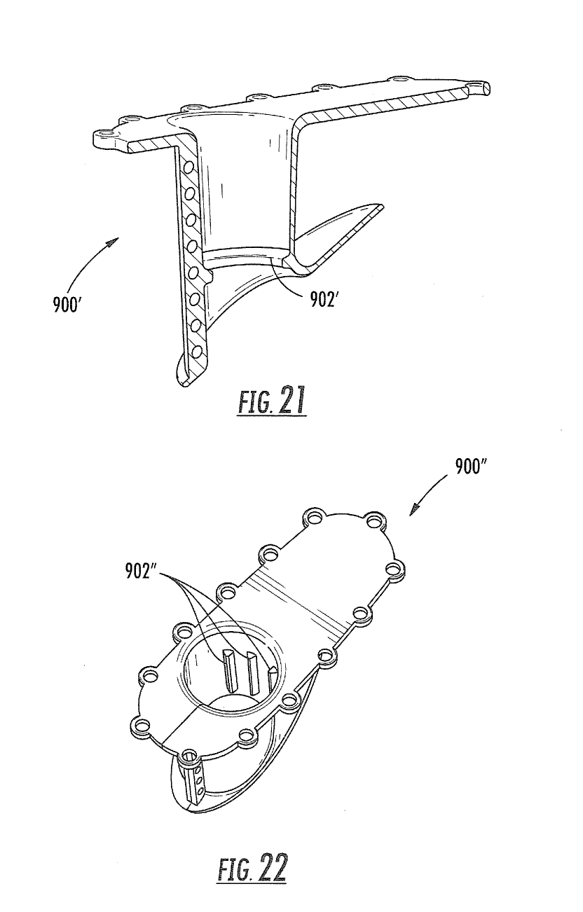

Another embodiment of a device for treating GERD, according to the present invention, is illustrated in FIGS. 20A-20B. FIG. 20A is a cross-sectional view of half of the device 900. The remaining, non-illustrated portion is similar to that of device 700. The illustrated device 900 is similar in construction and function to the device 700 of FIGS. 18A-18C with the exception of the inclusion of a raised portion or rib 902 located on the inner surface 922a of the housing 922. The rib 902 is configured to push on the esophagus when housing portion 923 elastically deforms when a force is applied to the plate 928 by the fundus F, as described above. The rib 902 facilitates closure of the esophagus at the GES and helps prevent regurgitation. FIG. 20B is a cross-sectional view of the device 900 taken along lines 20B-20B in FIG. 20A.

Although illustrated with a single rib 902, the device 900 may have multiple ribs in spaced-apart relationship and/or in various different orientations. The ribs may have various shapes and configurations without limitation. For example, in FIG. 21, the illustrated device 900′ includes a single rib 902′ in a substantially horizontal orientation. In FIG. 22, the illustrated device 900″ includes a plurality of ribs 902″ in substantially vertical orientations.

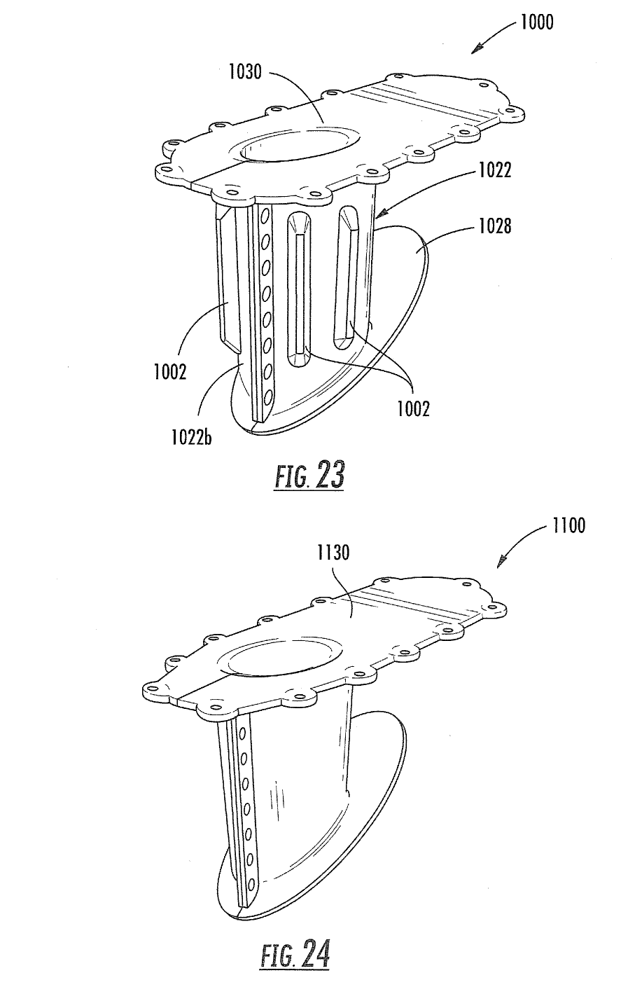

Another embodiment of a device for treating GERD, according to the present invention, is illustrated in FIG. 23. The illustrated device 1000 is similar in construction and function to the device 700 of FIGS. 18A-18C with the exception of stiffening ribs 1002 located on the outside surface 1022b of the housing 1022. Various numbers and configurations of ribs 1002 may be utilized, according to embodiments of the present invention. The ribs 1002 may have various lengths and may be staggered in relationship to each other. One or more of the ribs 1002 may also extend completely between the flange portion 1030 and the plate 1028. In addition, the ribs 1002 may be configured to facilitate the elastic deformation of the housing 1022 in a specific manner when a force is applied to the plate 1028 by the fundus F, as described above.

In some embodiments, the ribs in each of the illustrated embodiments of FIGS. 20A-20B and 21-23 may be reinforced, for example via insert molding wherein a reinforcing element is positioned within the material of the various devices.

Another embodiment of a device for treating GERD, according to the present invention, is illustrated in FIG. 24. The illustrated device 1100 is similar in construction and function to the device 700 of FIGS. 18A-18C with the exception of the enlarged configuration of the flange portion 1130. The enlarged flange portion 1130 has greater surface area than flange portion 730 and, because of the increased area, can facilitate attachment to a body cavity wall W, for example the diaphragm (D, FIG. 1) of a patient.

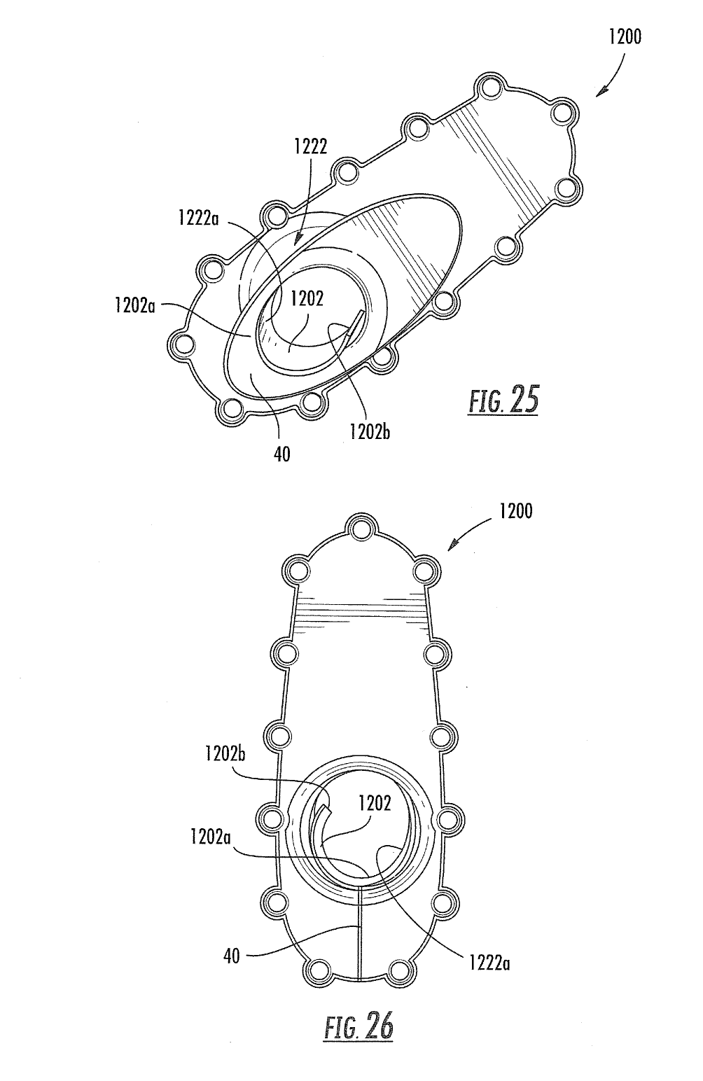

Another embodiment of a device for treating GERD, according to the present invention, is illustrated in FIGS. 25-26. The illustrated device 1200 is similar in construction and function to the device 700 of FIGS. 18A-18C with but also includes a flexible inner member 1202 attached to the housing inner surface 1222a. The flexible inner member 1202 has an end 1202a attached to the housing inner surface 1222a adjacent to a slit edge and has an opposite free end 1202b. When the device 1200 is stretched apart to wrap around an esophagus, the flexible inner member 1202 bridges the gap created when the device 1200 is stretched open, similar to the function of the tongue of a shoe. As the device 1200 is fastened closed, the flexible inner member free end 1202b slides along the housing inner surface 1222a on the other side of the slot 40, still bridging the gap. This allows the device 1200 to be closed to the desired position without leaving a gap.

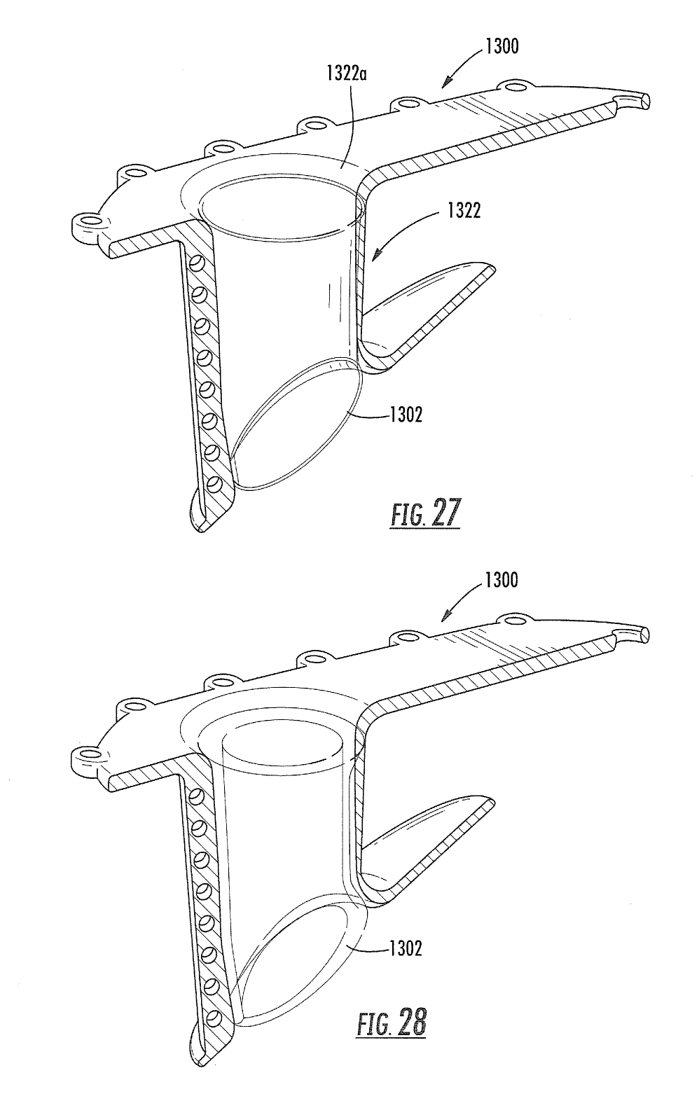

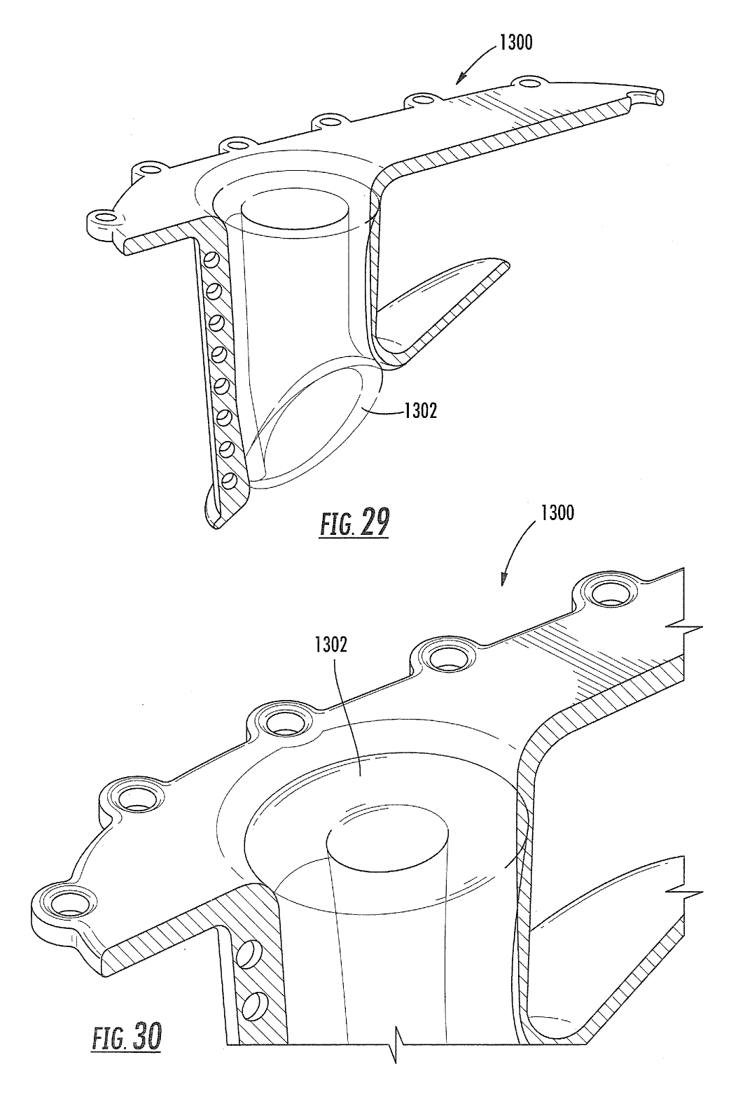

Another embodiment of a device for treating GERD, according to the present invention, is illustrated in FIGS. 27-32. The illustrated device 1300 is similar in construction and function to the device 700 of FIGS. 18A-18C with but also includes an inflatable liner 1302 attached and to and extending along the housing inner surface 1322a. The inflatable liner 1302 has a generally cylindrical configuration as illustrated. The inflatable liner 1302 can be expanded after the device 1300 is implanted around the esophagus of a patient to achieve the desired device size. FIGS. 28-31 illustrate the inflatable liner 1302 at increasingly expanded configurations.

The inflatable liner 1302 can be inflated to set the size thereof once, for example at implant. Alternatively, the inflated size of the liner 1302 can be adjustable, for example via an external fill port 1304 and fill tube 1306, illustrated in FIG. 32. The fill port 1304 could be located sub-cutaneous, intra-abdominal, or intra-gastric (inside the stomach). The fill port 1304 could also be used to fill the balloon 1302 once, and then sealed off and removed.

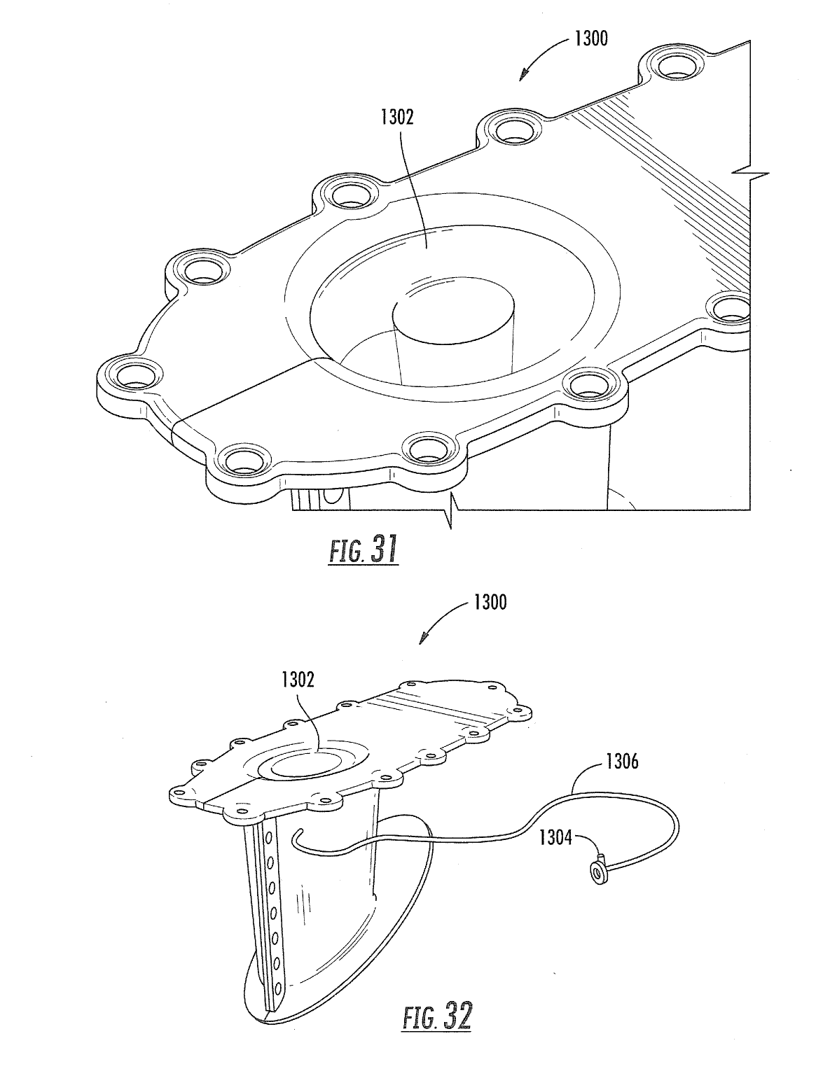

FIGS. 33-37 illustrate various types of fasteners for securing a GERD treatment device that has been wrapped around an esophagus, according to embodiments of the present invention. The illustrated GERD treatment device 700 is the same device illustrated in FIGS. 18A-18C. However, the fasteners described with respect to FIGS. 33-37 may be utilized in securing any of the various GERD treatment devices described herein.

In FIG. 33, the housing 722 of the illustrated GERD treatment device 700 includes a pair of enlarged rib portions 740a, 740b located at each free end of the housing 722 (i.e., at each slit edge). Rib portion 740b includes a respective plurality of spaced-apart apertures 742. Rib portion 740a includes a respective plurality of spaced-apart male members 744 that are configured to engage with the respective apertures 742 of rib portion 740b. Each male member 744 is configured to be secured within a respective aperture 742, thereby securing the rib portions 740a, 740b of the device 700 together around an esophagus. In some embodiments, the rib portions 740a, 740b may be rigid, semi-rigid, or flexible. In some embodiments, the rib portions 740a, 740b may be reinforced, for example via insert molding wherein a reinforcing element is positioned within the material that forms the rib portions 740a, 740b, as described above.

In FIG. 34, the housing 722 of the illustrated GERD treatment device 700 includes a pair of enlarged rib portions 740 located at each slit edge. Each rib portion 740 includes a respective plurality of spaced-apart apertures 742. A fastener 745 is configured to engage a respective aperture 742 in each rib portion 740 and secure the device 700 around an esophagus. Each fastener 745 includes a male member 746 and a female member 748. Each male member 746 is configured to be secured within a respective female member 748. In some embodiments, the rib portions 740 may be rigid, semi-rigid, or flexible. In some embodiments, the rib portions 740 may be reinforced, for example via insert molding wherein a reinforcing element is positioned within the material that forms the rib portions 740 and apertures 742, as described above. In some embodiments, the male and female members 746, 748 may be insert molded within the rib portions 740. In some embodiments, two or more of the male members 746 may be connected, and two or more of the female members 748 may be connected.

In FIG. 35, the housing 722 of the illustrated GERD treatment device 700 includes a pair of enlarged rib portions 740 located at each housing slit edge. Each rib portion 740 includes a respective plurality of spaced-apart apertures 742. A fastener 747 includes a first portion 754 and a second portion 756. The second portion 756 includes a plurality of spaced-apart apertures 760. The second portion 754 includes a respective plurality of spaced-apart male members 762 that are configured to engage with the respective apertures 760 of the second portion 754. Each male member 762 has a generally arrowhead shape and is configured to be secured within a respective aperture 760. The fastener 747 is configured to sandwich the respective rib portions 740 therebetween, thereby securing the device 700 around an esophagus.

In some embodiments, the first and second portions 754, 756 of the fastener 747 may be rigid, semi-rigid, or flexible. In some embodiments, the first and second portions 754, 756 of the fastener 747 may be reinforced, for example via insert molding wherein a reinforcing element is positioned within the material that forms the first and second portions 754, 756 of the fastener 747, as described above. In some embodiments, the first and second portions 754, 756 of the fastener 747 may be insert molded within the rib portions 740.

In the illustrated embodiment of FIG. 35, the housing 722 of the device 700 is surrounded by a reinforcing mesh 770. The reinforcing mesh 770 may be secured to the housing 722, for example via adhesive or fasteners. Alternatively, the reinforcing mesh 770 may be insert molded with the device 700. In some embodiments, the reinforcing mesh 770 is secured via the fastener 747.

Also illustrated in FIG. 35 is another fastener 780 that may be utilized. Each fastener 780 includes a male member 782 and a female member 784. Each male member 782 is configured to be secured within a respective female member 784 in a similar manner as described with respect to the embodiment of FIG. 34.

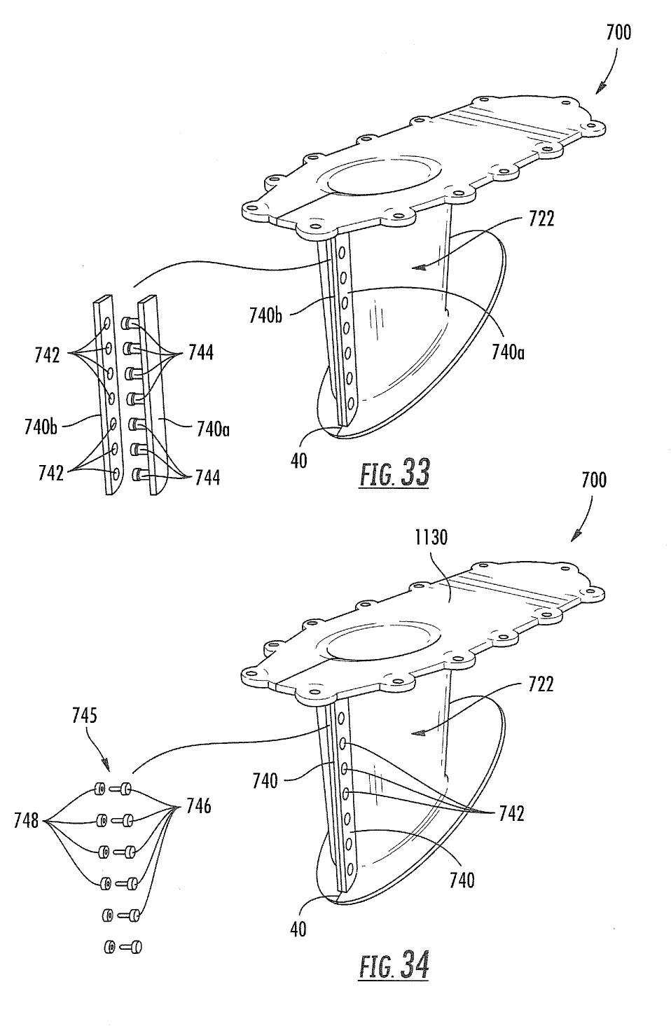

In FIG. 36, the housing 722 of the illustrated GERD treatment device 700 includes a pair of enlarged rib portions 740 located at each housing slit edge. A locking comb 790 is configured to grip the rib portions 740 and secure the device 700 around an esophagus. The locking comb 790 includes a pair of arms 792a, 792b that grip the rib portions 740. The locking comb 790 is expanded so that the arms 792a, 792b move away from each other. When released, the locking comb 790 is configured to bias the arms 792a, 792b toward is each other. Locking comb devices are well known in the art, and various types of locking combs may be utilized, without limitation. FIG. 37 is a partial section view illustrating the arms 792a, 792b of the locking comb 790 gripping the rib portions 740 of the device 700.

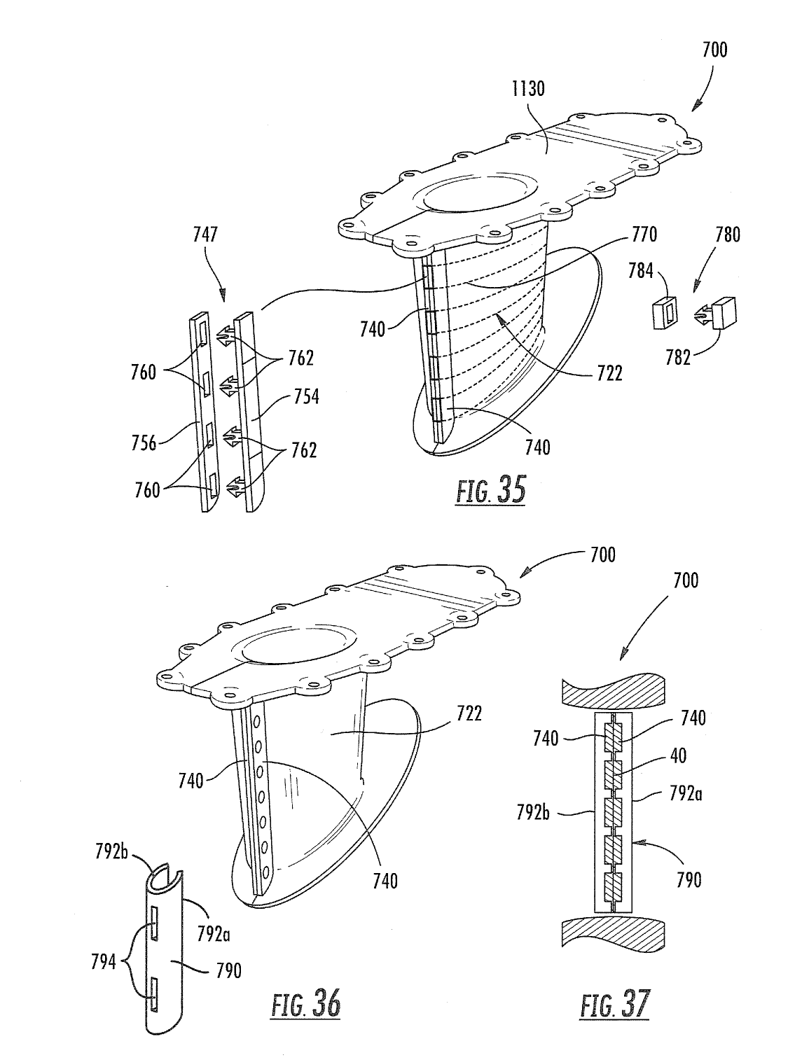

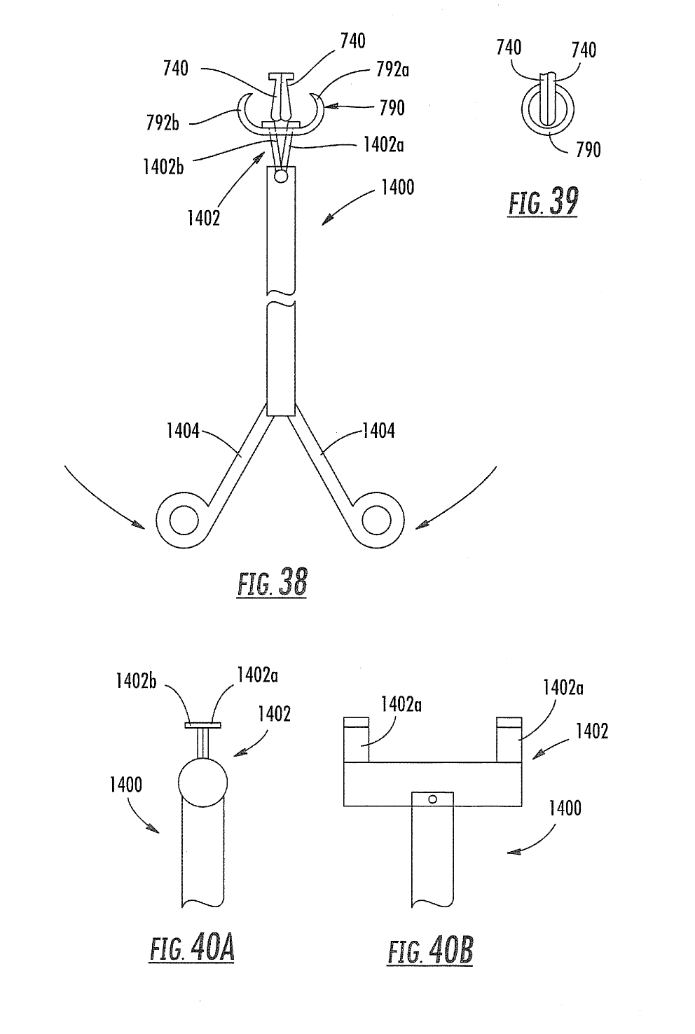

The illustrated locking comb 790 is expanded for insertion on a device 700 via a tool inserted within apertures 794. An exemplary tool 1400 for installing the locking comb 790 of FIGS. 36-37 is illustrated in FIGS. 38-39 and 40A-40B. The illustrated device 1400 includes a free end 1402 with two pairs of spaced-apart tips 1402a, 1402b configured to be inserted through the respective spaced-apart apertures 794 of the locking comb 790 (FIG. 36) and a pair of handles 1404. When the handles 1404 of the tool 1400 are moved towards each other (FIG. 38), the tips 1402a, 1402b move away from each other and cause the locking comb 790 to expand, thereby facilitating insertion of the locking comb 790 on the rib portions 740 of the device 700. When the handles 1404 of the tool 1400 are moved away from each other, the tips 1402a, 1402b move toward each other and the arms of the locking comb 790 bias toward each other so as to grip the rib portions 740 of the device 700 (FIG. 39). The tool 1400 may be utilized surgically, endoscopically, laparoscopically, and/or via a NOTES procedure.

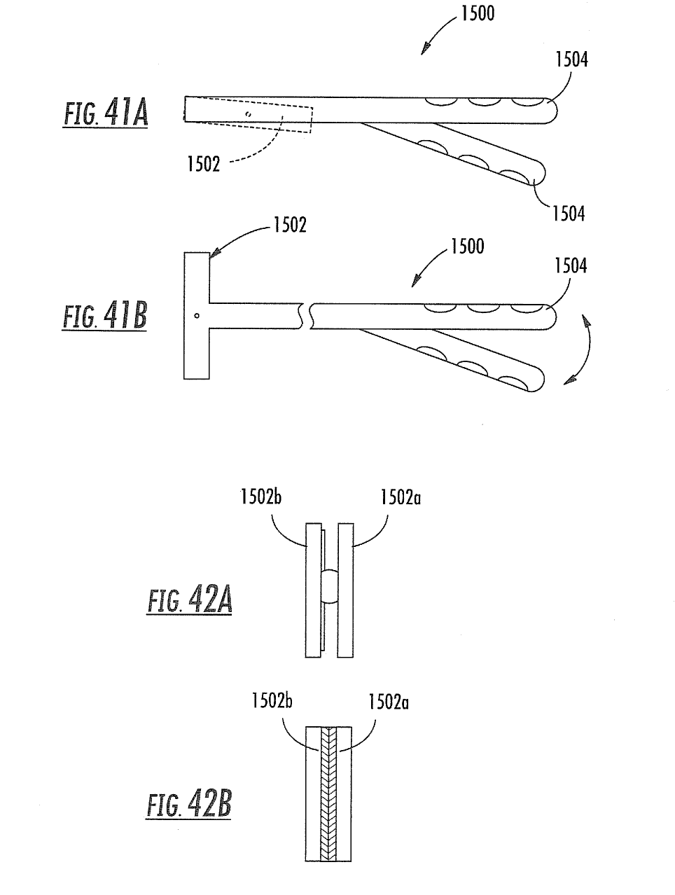

An exemplary tool 1500 for installing the fasteners illustrated in FIGS. 33-35 is illustrated in FIGS. 41A-41B and 42A-42B. The illustrated device 1500 includes a free end 1502 with a pair of jaws 1502a, 1502b and a pair of handles 1504. The free end 1502 is configured to pivot to facilitate insertion of the tool 1500 within a patient. When the handles 1504 are moved towards each other, the jaws 1502, 1502b are pressed together (FIG. 42B). When, for example, fastener 747 is inserted between the jaws 1502a, 1502b, the first and second portions 754, 756 of the fastener 747 can be pressed together to secure the GERD treatment device 700, as described above. The tool 1500 may be utilized surgically, endoscopically, laparoscopically, and/or via a NOTES procedure.

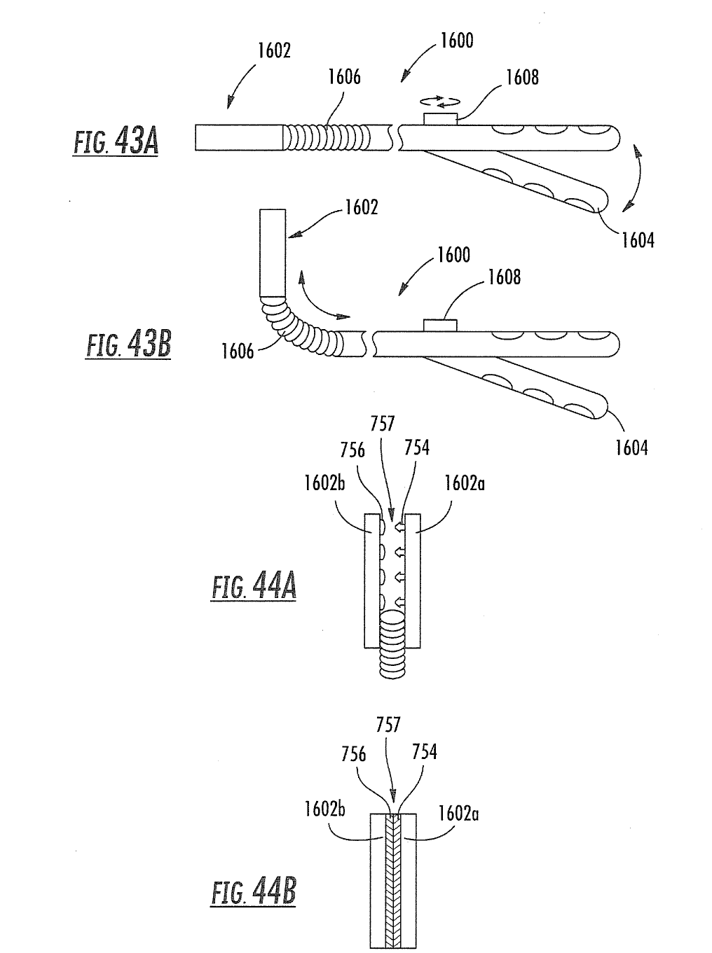

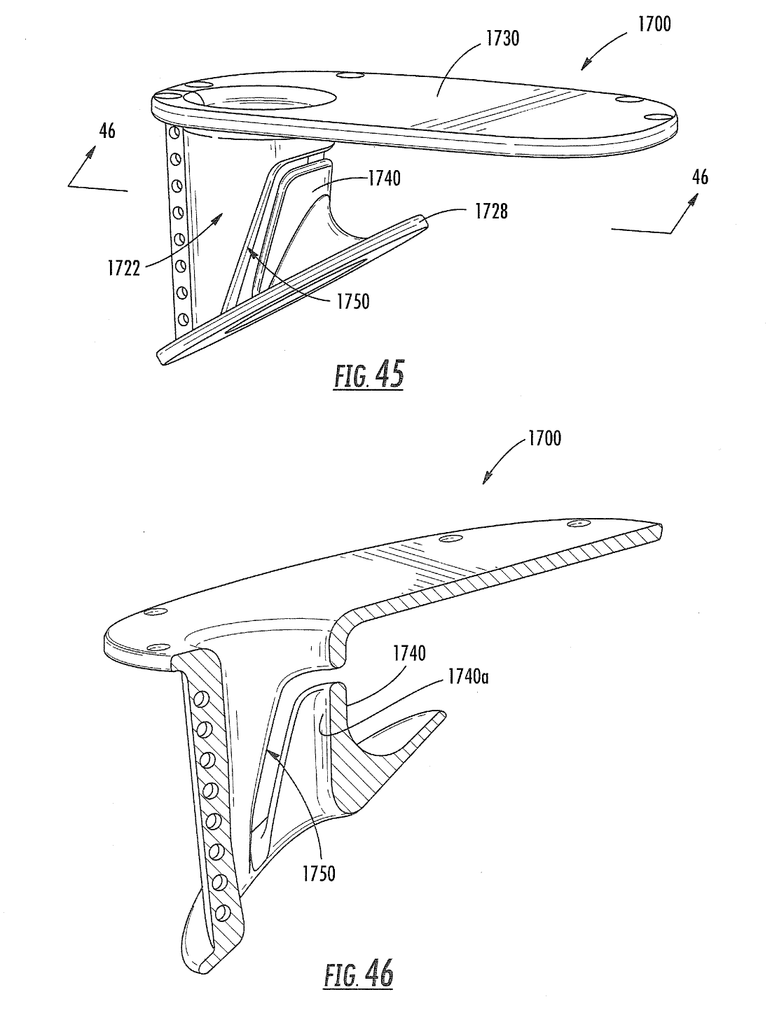

Another exemplary tool 1600 for installing the fasteners illustrated in FIGS. 33-35 is illustrated in FIGS. 43A-43B and 44A-44B. The illustrated device 1600 includes a free end 1602 with a pair of jaws 1602a, 1602b, a pair of handles 1604, and an articulating neck portion 1606. The free end 1602 is configured to be manipulated into various positions within a patient via the articulating neck 1606. When the handles 1604 are moved towards each other, the jaws 1602a, 1602b are pressed together (FIG. 44B). When, for example, fastener 747 is inserted between the jaws 1602a, 1602b, the first and second portions 754, 756 of the fastener 747 can be pressed together to secure the GERD treatment device 700, as described above. The tool 1600 may be utilized surgically, endoscopically, laparoscopically, and/or via a NOTES procedure.

Another embodiment of a device for treating GERD, according to the present invention, is illustrated in FIGS. 45-47. The illustrated device 1700 is similar in construction and function to the device 400 of FIGS. 15A-15B. However, the illustrated device 1700 includes an aperture 1750 in a side of the housing 1722 thereof. The plate 1728 is connected to a flap 1740 and the flap 1740 is configured to move through the aperture 1750 and into the housing 1722 when a force is exerted on the plate 1728 by the fundus F, as described above. Movement of the flap 1750 into the housing 1722 via aperture 1750 constricts an esophagus around which the device 1700 is implanted.

In some embodiments the flap 1750 is biased into the housing 1722 so as to continually support/close the LES and is opened (pushed back) by the passage of food down the esophagus during swallowing.

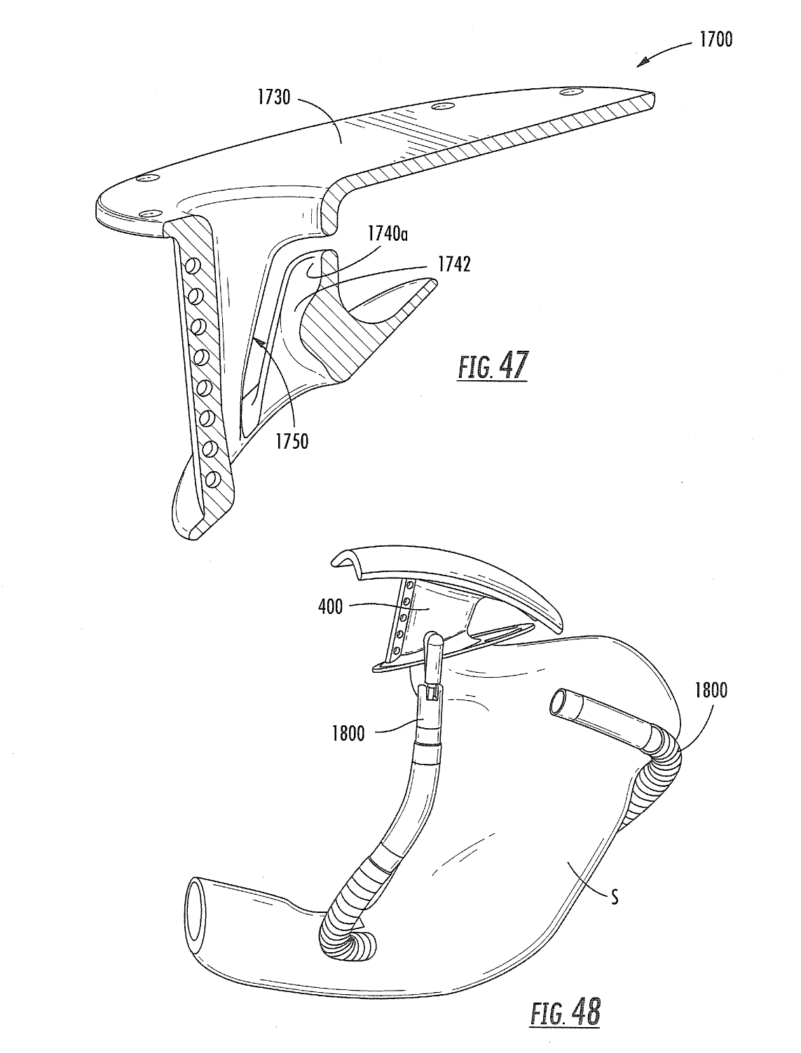

In some embodiments, the flap inner surface 1740a has a generally smooth configuration, as illustrated in FIG. 46. In other embodiments, the flap inner surface 1740a includes one or more raised portions or ribs 1742, as illustrated in FIG. 47. A rib on the inner surface 1740a helps constrict an esophagus when the flap 1750 moves into the housing 1722 via aperture 1750. In some embodiments, the rib 1742 may be an interchangeable component that can be inserted and removed from the flap 1750. Moreover, the rib 1742 may be adjustable to allow a surgeon to adjust the amount of closure on the esophagus. An interchangeable rib 1742 can be snapped, glued, stapled, sutured, or otherwise fixed in place, and may be formed from the same or similar material as the flap 1750 and/or device 1700. In some embodiments, an interchangeable rib 1742 may have a tab or other member connected thereto that extends through the flap 1750 and that facilitates insertion and removal of the rib to and from the flap 1750.

In all of the embodiments described herein, various aspects of each GERD treatment device (10, 100, 200, 300, 400, 500, 600, 700, 800, 900, 900′, 900″, 1000, 1100, 1200, 1300, 1700) may vary from patient to patient. For example, the size and orientation of the various flange portions may vary, the size and orientation of the various plates may vary, the cross sectional areas of the apertures may vary, etc. Moreover, the angle of the various plates relative the respective device housings may vary. In addition, the wall thickness of the various devices may be different at various locations to achieve the desired flexibility in that specific area.

### Materials

GERD treatment devices, according to embodiments of the present invention, may be formed from various materials including, but not limited to polymeric materials and metals, including polymeric meshes and fabrics and metallic meshes and fabrics. Exemplary fabrics may include woven fabrics, nonwoven fabrics, a knitted fabrics, braid fabrics, etc. Devices, according to embodiments of the present invention, may be formed from antiadhesive materials or non tissue adhering materials.

GERD treatment devices, according to embodiments of the present invention, may be formed from or coated with materials having various combinations of silicone and metal, including composites and hybrids and blends thereof.

GERD treatment devices, according to embodiments of the present invention, may be formed in various ways including, but not limited to, molding, insert molding, casting, machining, etc.

In addition, GERD treatment devices, according to embodiments of the present invention, may be formed or coated with polymeric material. For example, one or more portions of the devices may be formed from (or coated with) polymeric material. Exemplary polymeric materials that may be utilized include, but are not limited to, elastomers, rubbers (e.g., nitrile, latex, etc.), polyurethanes (e.g., ChronoFlex® polyurethane, etc.), polyolefins, poly(meth)acrylates, polyesters (e.g., Dacron® polyester), ePTFE fabric (e.g., GoreTex® fabric or others), polyamides, polyvinyl resins, silicon resins, polycarbonates, polyfluorocarbon resins, synthetic resins, polystyrene, nylon fabrics, silicone, bio-absorbable materials (e.g., PLLA, PGA, PCL, poly-amhydride etc).

Moreover, devices, according to some embodiments of the present invention, may be formed of a composite of compliant, semi-compliant and/or non-compliant materials which give different regions of the device different degrees of compliance so as to allow/limit expansion of the device in various locations. For example, it may be desirable to provide a device with elastic portions so as to prevent occlusion in the event a large piece of food is ingested. Varying degrees of compliance may also be built into the device by varying the cross-sectional thickness of the device in different regions thereof. In some embodiments, the device material may be coated with a lubricious, bio-compatible, chemically inert material, such as paraleyne, to reduce friction with a stomach.

In some embodiments, polymeric material utilized is non-erodible (or the device has a non-erodible coating), although in other embodiments it may be desirable for the polymeric material to be erodible (or the device may have an erodible coating). Exemplary erodible materials include, but are not limited to, surgical gut, silk, cotton, poly(hydroxybutyrate), polycarbonate, polyacrylate, polyanhydride, poly(ortho esters), poly(phosphoesters), polyesters, polyamides, polyphosphazenes, poly(p-dioxane), poly(amino acid), polyglactin, erodable hydrogels, collagen, chitosan, poly(lactic acid), poly(L-lactic acid), poly(D,L-lactic acid), poly(glycolic acid), poly(D-lactic-co-glycolic acid), poly(L-lactic-co-glycolic acid), poly (D,L-lactic-co-glycolic acid), poly(C-caprolactone), poly(valerolactone), poly(hydroxy butyrate), poly(hydrovalerate), polydioxanone, poly(propylene fumarate), poly(ethyleneoxide)-poly(butylenetetraphthalate), poly(lactic acid-co-lysine), poly(lactic acid-co-trimethylene carbonate), poly(L-lactic acid) and poly(C-caprolactone) copolymers, and blends thereof. Exemplary non-erodible materials include, but are not limited to, fluoropolymers, polyesters, PET, polyethylenes, polypropylenes, etc., and/or ceramics, such as hydroxyapetite.

Exemplary metallic materials include titanium and platinum, metal alloys, such as stainless steel, nickel-titanium, and cobalt-chromium, etc.

One or more portions of the various GERD treatment device embodiments described herein may be formed from shape memory material, such as nitinol.

GERD treatment devices, according to some embodiments of the present invention, may include various pharmacological agents. In general, pharmacological agents suitable for inclusion in materials and/or coatings (and according to embodiments of the present invention) include, but are not limited to, drugs and other biologically active materials, and may be intended to perform a variety of functions, including, but not limited to: anti-infection treatment, anti-inflammatory treatment, and the prevention of smooth muscle cell growth, migration, proliferation within a vessel wall. Pharmacological agents may include antineoplastics, antimitotics, antiinflammatories, antiproliferatives, antibiotics, and antiallergic substances as well as combinations thereof. Examples of antineoplastics and/or antimitotics include paclitaxel (cytostatic and ant-inflammatory) and its analogs and all compounds in the TAXOL® (Bristol-Myers Squibb Co., Stamford, Conn.) family of pharmaceuticals, docetaxel (e.g., TAXOTERE® from Aventis S. A., Frankfurt, Germany) methotrexate, azathioprine, vincristine, vinblastine, fluorouracil, doxorubicin hydrochloride (e.g., ADRIAMYCIN® from Pharmacia & Upjohn, Peapack N.J.), and mitomycin (e.g., MUTAMYCIN® from Bristol-Myers Squibb Co., Stamford, Conn.). Examples of antiinflammatories include Sirolimus and its analogs (including but not limited to Everolimus and all compounds in the Limus family of pharmaceuticals), glucocorticoids such as dexamethasone, methylprednisolone, hydrocortisone and betamethasone and non-steroidal antiinflammatories such as aspirin, indomethacin and ibuprofen. Examples of cytostatic or antiproliferative agents or proliferation inhibitors include everolimus, actinomycin D, as well as derivatives and analogs thereof (manufactured by Sigma-Aldrich, Milwaukee, Wis.; or COSMEGEN® available from Merck & Co., Inc., Whitehouse Station, N.J.), angiopeptin, angiotensin converting enzyme inhibitors such as captopril (e.g., CAPOTEN® and CAPOZIDE® from Bristol-Myers Squibb Co., Stamford, Conn.), cilazapril or lisinopril (e.g., Prinivilo and PRINZIDE® from Merck & Co., Inc., Whitehouse Station, N.J.); calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name MEVACOR® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), and nitric oxide. An example of an antiallergic agent is permirolast potassium. Other therapeutic substances or agents that may be used include alphainterferon, anti-Tumor Necrosis Factor (TNF) α, genetically engineered epithelial cells, and dexamethasone.

### Implantation

Implantation of the various GERD treatment device embodiments may be performed using various procedures including, but not limited to, surgery, laparoscopy, endoscopy, and Natural Orifice Translumenal Endoscopic Surgery (NOTES). In a laparoscopic procedure, surgeons use small incisions (e.g., ¼ to ½ inch) to enter the abdomen through cannulas (narrow tube-like instruments). The laparoscope, which is connected to a tiny video camera, is inserted through the small cannula. A picture is projected onto a monitor giving the surgeon a magnified view of the stomach and other internal organs. Five to six small incisions and cannulas are placed for use of specialized instruments to implant the various GERD treatment devices (10, 100, 200, 300, 400, 500, 600, 700, 800, 900, 900′, 900″, 1000, 1100, 1200, 1300, 1700), according to embodiments of the present invention. The entire operation is performed inside the abdomen after expanding the abdomen with carbon dioxide (CO₂) gas. The CO₂ gas is removed at the completion of the operation. The abdominal space may also be expanded using other techniques including “gasless” laparoscopy.

In an endoscopic procedure, an endoscope is gently passed through the mouth, down the esophagus, and into the stomach and duodenum. The endoscope is connected to a tiny video camera from which a picture is projected onto a monitor giving the surgeon a magnified view of the stomach and other internal organs. Specialized instruments to implant the various GERD treatment devices (10, 100, 200, 300, 400, 500, 600, 700, 800, 900, 900′, 900″, 1000, 1100, 1200, 1300, 1700), according to embodiments of the present invention, are passed through the endoscope.

A NOTES procedure is a new type of surgical procedure currently being studied at research hospitals and facilities around the world. NOTES procedures have been developed because: patient recovery time can be reduced because the procedures are less invasive than conventional surgical procedures, because patients experience less physical discomfort than with traditional procedures, and because patients have virtually no visible scarring following this type of surgery. According to embodiments of the present invention, a doctor inserts a tube down the esophagus, makes a small incision in the stomach or digestive tract to gain access to the abdominal cavity and implants the various GERD treatment devices (10, 100, 200, 300, 400, 500, 600, 700, 800, 900, 900′, 900″, 1000, 1100, 1200, 1300, 1700) of the present invention. FIG. 48 illustrates the implantation of the GERD treatment device 400 of FIGS. 15A-15B around an esophagus via a NOTES device inserted down the esophagus of a patient, and wherein arms or portions 1800 of the NOTES device are extending through the stomach wall.

In the drawings and specification, there have been disclosed typical preferred embodiments of the invention and, although specific terms are employed, they are used in a generic and descriptive sense only and not for purposes of limitation, the scope of the invention being set forth in the following claims.

That which is claimed:

1. A device for treating gastroesophageal reflux disease (GERD) and/or hiatal hernia that is configured to be positioned around the esophagus of a patient adjacent to the fundus, the device comprising a pair of mating sections, each mating section including:
a proximal end portion with an arcuate interior opening;
a distal end portion with an arcuate interior opening and an exterior plate portion; and
a body portion extending from the proximal end portion to the distal end portion;
wherein the pair of mating sections are configured to surround the esophagus at the gastro-esophageal junction thereof and to be secured together to form a tubular housing with a proximal orifice, a distal orifice, and a plate adjacent the distal orifice, and wherein a portion of the housing adjacent the distal orifice is configured to elastically deform to constrict the esophagus when a force is applied to the plate by the fundus.

2. The device of claim 1, wherein the proximal orifice defines a first axial direction, and wherein the distal orifice defines a second axial direction that is different from the first axial direction.

3. The device of claim 2, wherein the plate portion is elongated in a direction substantially transverse to the first and second axial directions.

4. The device of claim 2, wherein the plate portion is elongated in a direction substantially orthogonal to the second axial direction.

5. The device of claim 1, wherein the proximal orifice is larger than the distal orifice.

6. The device of claim 1, wherein the proximal orifice has a generally circular configuration.

7. The device of claim 1, wherein the distal orifice has a generally oval configuration.

8. The device of claim 1, wherein a size of the distal orifice and/or proximal orifice is adjustable.

9. The device of claim 8, wherein the size of the distal orifice and/or proximal orifice is transabdominally adjustable mechanically, magnetically, pneumatically, hydraulically, and/or telemetrically via RF energy or ultrasound.

10. The device of claim 1, wherein the mating sections are hingedly connected.

11. The device of claim 1, wherein the proximal end portion of each mating section comprises a flange portion configured to be attached to a body cavity wall.

12. The device of claim 9, wherein each flange portion comprises a plurality of spaced-apart apertures.

13. The device of claim 1, wherein the mating sections comprise polymeric material.

14. The device of claim 13, wherein the polymeric material is erodible.

15. The device of claim 13, wherein the polymeric material is non-erodible.

16. The device of claim 1, wherein the mating sections comprise drug eluting material.

17. The device of claim 1, wherein the mating sections comprise material configured to stimulate tissue growth.

18. The device of claim 1, wherein the mating sections comprise shape memory material.

19. The device of claim 18, wherein the shape memory material comprises nitinol.

20. The device of claim 18, wherein the shape memory material comprises a shape memory polymer.

21. The device of claim 1, wherein the mating sections comprise a mesh.

22. The device of claim 1, wherein the mating sections comprise metal.

23. The device of claim 1, wherein the mating sections are secured together via adhesive, via one or more snap connectors, and/or via one or more fasteners.

24. A method of treating gastroesophageal reflux disease (GERD) and/or hiatal hernia, comprising:
securing a pair of mating sections around the esophagus of a patient at the gastro-esophageal junction thereof and adjacent the fundus, wherein each mating section includes:
a proximal end portion with an arcuate interior opening;
a distal end portion with an arcuate interior opening and an exterior plate portion; and
a body portion extending from the proximal end portion to the distal end portion;
wherein the pair of mating sections form a tubular housing with a proximal orifice, a distal orifice, and a plate adjacent the distal orifice, and wherein a portion of the housing adjacent the distal orifice is configured to elastically deform so as to constrict the esophagus when a force is applied to the plate by the fundus; and
attaching one or more of the proximal end portions to a body cavity wall of the patient.

25. The method of claim 24, wherein the mating sections are secured around the esophagus such that the plate is in contacting relationship with the fundus.

26. The method of claim 24, wherein the mating sections are secured via a laparoscopy tool, via an endoscope, and/or via a NOTES device inserted down the esophagus of a patient.

27. The method of claim 24, wherein the mating sections are secured via a surgical procedure.

28. The method of claim 24, wherein the proximal end portions are attached to the body cavity wall via one or more surgical clips, one or more sutures, one or more staples, or via adhesive.

29. The method of claim 24, wherein the mating sections are secured together via one or more snap connectors, via one or more fasteners, and/or via adhesive.

30. The method of claim 24, wherein the mating sections are hingedly connected.

31. The method of claim 24, wherein the proximal orifice defines a first axial direction, and wherein the distal orifice defines a second axial direction that is different from the first axial direction.

32. The method of claim 31, wherein the plate portion is elongated in a direction substantially transverse to the first and second axial directions.

33. The method of claim 31, wherein the plate portion is elongated in a direction substantially orthogonal to the second axial direction.

34. The method of claim 24, wherein the proximal orifice is larger than the distal orifice.

35. The method of claim 24, wherein the proximal orifice has a generally circular configuration.

36. The method of claim 24, wherein the distal orifice has a generally oval configuration.

37. The method of claim 24, wherein the proximal end portion of each mating section comprises a flange portion configured to be attached to a body cavity wall.

38. The method of claim 37, wherein each flange portion comprises a plurality of spaced-apart apertures.

39. The method of claim 24, wherein the mating sections comprise polymeric material.

40. The method of claim 24, wherein the polymeric material is erodible.

41. The method of claim 24, wherein the polymeric material is non-erodible.

42. The method of claim 24, wherein the mating sections comprise drug eluting material.

43. The method of claim 24, wherein the mating sections comprise material configured to stimulate tissue growth.

44. The method of claim 24, wherein the mating sections comprise shape memory material.

45. The method of claim 44, wherein the shape memory material comprises nitinol.

46. The method of claim 44, wherein the shape memory material comprises a shape memory polymer.

47. The method of claim 24, wherein the mating sections comprise a mesh.

48. The method of claim 24, wherein the mating sections comprise metal.

49. A device for treating gastroesophageal reflux disease (GERD) and/or hiatal hernia that is configured to be positioned around the esophagus of a patient adjacent to the fundus, comprising:
a tubular housing with opposite proximal and distal orifices; and
a plate adjacent the distal orifice that extends outwardly from the housing;
wherein a portion of the housing adjacent the distal orifice is configured to elastically deform to constrict the esophagus when a force is applied to the plate by the fundus.

50. The device of claim 49, wherein the distal orifice is smaller than the proximal orifice.

51. The device of claim 49, wherein a size of the distal orifice and/or proximal orifice is adjustable.

52. The device of claim 51, wherein the size of the distal orifice and/or proximal orifice is transabdominally adjustable mechanically, magnetically, pneumatically, hydraulically, and/or telemetrically via RF energy or ultrasound.

53. The device of claim 49, wherein the housing has a longitudinally extending slit with a pair of opposed slit edges facing each other, wherein the slit allows the housing to be spread open and wrapped around an esophagus.

54. The device of claim 51, wherein the housing slit edges are configured to be joined together.

55. The device of claim 54, further comprising at least one fastener configured to secure the housing slit edges together.

56. The device of claim 55, wherein the at least one fastener comprises first and second portions, and wherein the first and second portions are secured together with the housing slit edges sandwiched therebetween.

57. The device of claim 49, wherein the plate includes at least one slot formed therein.

58. The device of claim 49, wherein the housing comprises an inner surface, and wherein the inner surface comprises at least one raised portion extending outwardly therefrom.

59. The device of claim 58, wherein the at least one raised portion comprises at least one elongated member.

60. The device of claim 58, wherein the at least one raised portion comprises a plurality of spaced-apart elongated members.

61. The device of claim 49, wherein the housing comprises an outer surface, and wherein the outer surface comprises at least one raised portion extending outwardly therefrom.

62. The device of claim 61, wherein the at least one raised portion comprises at least one elongated member.

63. The device of claim 61, wherein the at least one raised portion comprises a plurality of spaced-apart elongated members.

64. The device of claim 49, further comprising a flange adjacent the proximal orifice that is configured to be attached to a body cavity wall.

65. The device of claim 64, wherein the flange has an elongated configuration that extends outwardly from the housing;

66. The device of claim 64, wherein the flange comprises a plurality of spaced-apart apertures.

67. The device of claim 49, wherein the housing comprises an inner surface, and further comprising a flexible member having a free end and an opposite end attached to a portion of the housing inner surface adjacent a slit edge, wherein the flexible member overlies a portion of the slit.

68. The device of claim 49, further comprising an inflatable liner secured within the housing, wherein the liner can be inflated to a predetermined size around the esophagus.

69. The device of claim 68, further comprising a fill tube in communication with the inflatable liner, and wherein the fill tube terminates at a fill port.

70. The device of claim 49, wherein the housing comprises an outer surface, and further comprising a reinforcing mesh secured to the housing outer surface.

71. A method of treating gastroesophageal reflux disease (GERD) and/or hiatal hernia, comprising:
implanting a device around a portion of the esophagus of a patient at the gastro-esophageal junction thereof and adjacent the fundus, wherein the device includes:
a tubular housing with opposite proximal and distal end portions, wherein the housing has a longitudinally extending slit that allows the housing to be spread open and wrapped around an esophagus; and
a plate adjacent the distal orifice that extends outwardly from the housing, and wherein a portion of the housing adjacent the distal orifice is configured to elastically deform to constrict the esophagus when a force is applied to the plate by the fundus; and
attaching the proximal end portion to a body cavity wall of the patient.

72. A device for treating gastroesophageal reflux disease (GERD) and/or hiatal hernia that is configured to be positioned around the esophagus of a patient adjacent to the fundus, comprising:
a tubular housing with opposite proximal and distal orifices;
an aperture formed within a portion of the housing;
a plate pivotally engaged with the housing adjacent the distal orifice, wherein the plate extends outwardly from the housing and is pivotable between a first position and second position; and
a flap attached to the plate, wherein the flap is configured to extend within the housing to constrict the esophagus when the plate is moved to the second position by the fundus.

73. The device of claim 72, wherein the flap comprises a raised portion that contacts the esophagus when the plate is moved to the second position.
